(19) United States
(12) Patent Application Publication
(10) Pub. No.: US 2026/0293937 A1
(43) Pub. Date: Oct. 1, 2026

(54) SHRIMP FEED AND METHODS OF USE THEREOF

(71) Applicant: Organicin Scientific, Inc., Wilmington, DE (US)

(72) Inventors: Griffin O'Driscoll, Northborough, MA (US); Mathew Mitchell, Philadelphia, PA (US); Margaret Riley, Amherst, MA (US)

(21) Appl. No.: 19/478,582

(22) PCT Filed: Apr. 26, 2024
(86) PCT No.: PCT/US2024/026564

§ 371 Date: Oct. 24, 2025

Related U.S. Application Data
(60) Provisional application No. 63/462,067, filed on Apr. 26, 2023.

(51) Int. Cl. A23K 50/80; A23K 10/22; A23K 10/30; A23K 20/105; A23K 20/147; A23K 20/158; A23K 20/163; A23K 20/174; A23K 20/195; A23K 20/22; A23K 20/24; A23K 20/26; A61K 9/00; A61K 31/198; A61K 38/16; A61K 47/12; A61K 47/38; A61P 31/04

(52) U.S. Cl. CPC A23K 50/80; A23K 10/22; A23K 10/30; A23K 20/105; A23K 20/147; A23K 20/158; A23K 20/163; A23K 20/174; A23K 20/195; A23K 20/22; A23K 20/24; A23K 20/26; A61K 9/0056; A61K 31/198; A61K 38/164; A61K 47/12; A61K 47/38; A61P 31/04

(57) ABSTRACT

The present disclosure relates to methods and compositions for preventing or treating a disease or one or more symptoms thereof caused by pathogenic Vibrio in a shrimp in need thereof. Compositions of the disclosure include a bacteriocin (e.g., Nisin) and shrimp feed.

### CROSS REFERENCE TO RELATED APPLICATIONS

This application claims the benefit of and priority to U.S. Provisional Application No. 63/462,067 filed Apr. 26, 2023, the entire disclosure of which is hereby incorporated by reference in its entirety for all purposes.

### BACKGROUND

Aquaculture is the fastest growing sector of food production in the world. The Food and Agriculture Organization (“FAO”) of the United Nations reports that aquaculture accounts for almost 50% of the world's food fish. Aquaculture has the greatest potential to meet the growing demand of aquatic food that will arise from an expected population increase of 1.5 billion people in 2020. In order to maintain the current per capita consumption, an additional 40 million tons of aquatic food will be required by 2030. Mounting scientific evidence indicates that dramatic declines in many natural fish stocks are occurring. Capture fisheries are not capable of providing fish to an additional 1.5 billion people, considering FAO already lists 89% of the ocean's wild fish stocks as moderately, fully or over-exploited.

In addition to meeting global consumption needs, aquaculture, particularly shrimp farming, has become an increasingly important economic activity. In recent years, high value and market demand by mainly affluent consumers in developed countries has led to rapid expansion of shrimp aquaculture throughout Asia and Latin America. In 1999, shrimp aquaculture only represented about 2.6% of total global aquaculture, but accounted for 16.5% of the total revenue at a value of about $6.7 billion. Considerable private and public sector investment has induced an annual average increase in cultured shrimp production of about 5-10% since the 1990s.

Shrimp farming is one of the most profitable and fastest-growing segments of the aquaculture industry. Global farmed shrimp production has grown more than 100-fold (by weight) in less than two decades; from under 10,000 metric tons (MT) produced by fewer than a dozen countries in the early 1970s to over 1 million MT (MMT) by the late 1990s. Worldwide sale of shrimp is estimated to be a 10 billion dollar industry. In 2006, aquafeeds used 68% of the world's supply of fish meal (3.724 million metric tons) and 88.5% of fish oil (835,000 million metric tons). Aquafeeds production is expected to double within 10 years. Researchers began looking for fish meal substitutes in earnest after an El Nino event (e.g., climate pattern event) in the 1970s drastically reduced fish meal production and sent prices to historic highs. Protecting against catastrophic events was one goal, as well as protecting fish populations by not harvesting as many for fish meal. While fish meal prices have remained relatively stable, prices are about 50% higher than just a few years ago. At the same time, the prices of corn, soybeans, wheat, and rice have increased.

Vibriosis is the term used to refer to a multitude of infections caused by bacteria belonging to the genus Vibrio (e.g., Vibrio parahaemolyticus, Vibrio alginolyticus, Vibrio vulnificus, Vibrio cholera, Vibrio harveyi, Vibrio fischeri, Vibrio anguillarum, Vibrio spledidus, Vibrio nerves, Vibrio tubiashii, Vibrio campbellii, Vibrio aestuarianus, Vibrio pelagius, Vibrio mediterranei, Vibrio diazotrophicus, Vibrio damselae, Vibrio mimicus, and Vibrio cholera). The direct economic implications of vibriosis can be devastating, particularly when acute outbreaks occur. The major species causing vibriosis in shrimp are Vibrio parahaemolyticus, Vibrio alginolyticus, Vibrio vulnificus, Vibrio cholera, and Vibrio harveyi. If vibriosis occurs in the hatchery, the loss can be up to 100%. For example, 22% of shrimp produced are lost annually due to disease, such as acute hepatopancreatic necrosis disease (AHPND). Additionally, with the demand for seafood protein production, complementary industries, such as shrimp feed, are also growing.

In shrimp production, the largest importers have banned antibiotic residues, which is pertinent as shrimp lack an adaptive immune system, thereby requiring the production of shrimp to be performed without antibiotics. Therefore, an outstanding need exists for shrimp feed compositions and methods of manufacturing thereof, which provide high efficacy against the causative agents of shrimp disease, such as AHPND.

### SUMMARY

The present invention pertains to the prevention of disease and reduction of pathogenic Vibrio of shrimp through the use of shrimp feed including a bacteriocin.

The present disclosure provides compositions comprising: a bacteriocin, wherein the bacteriocin comprises Nisin; and shrimp feed, wherein the composition prevents or treats a disease or one or more symptoms thereof caused by pathogenic Vibrio in a shrimp in need thereof.

The present disclosure also provides methods of preventing or treating a disease or one or more symptoms thereof caused by pathogenic Vibrio in a shrimp in need thereof, the method comprising administering a composition comprising: a bacteriocin, wherein the bacteriocin comprises Nisin; and shrimp feed.

The present disclosure also provides methods of preventing or treating a disease or one or more symptoms thereof caused by pathogenic Vibrio in a shrimp in need thereof, the method comprising administering a composition comprising: a bacteriocin, wherein the bacteriocin comprises Nisin: shrimp feed; and EDTA.

The present disclosure also provides methods of reducing a concentration of pathogenic Vibrio in a shrimp in need thereof, the method comprising administering a composition comprising: a bacteriocin, wherein the bacteriocin comprises Nisin; and shrimp feed.

The present disclosure also provides methods of reducing a concentration of pathogenic Vibrio in a shrimp in need thereof, the method comprising administering a composition comprising: a bacteriocin, wherein the bacteriocin comprises Nisin; shrimp feed; and EDTA.

In some embodiments, the shrimp feed comprises: one or more of proteins, amino acids, carbohydrates and/or starches, vitamins and/or minerals, fatty acids and/or phospholipids, non-nutrient additives, and combinations thereof.

In some embodiments, the proteins comprise: fish meal, soybean meal, shrimp meal, sardine meal, poultry meal, squid meal, corn gluten meal, krill meal, insect meal, and protein from a single-cell source; the amino acids comprise: methionine, threonine, lysine, arginine, valine, histidine, phenylalanine, and tryptophan; c. the carbohydrates and/or starches comprise: wheat flour, wheat gluten meal, corn, and rice: d. the vitamins and/or minerals comprise: pre-mix, Ca(H₂PO₄)₂ with vitamin C, choline chlorine with chloride, salt, potassium chloride, calcium carbonate, magnesium sulfate, and monosodium phosphate; e. the fatty acids and/or phospholipids comprise: fish oil, tuna oil, squid oil, krill oil, algae oil, soy lecithin oil, and vegetable oil; and/or f. the non-nutrient additives comprise: a binder, soy lecithin, and a mold inhibitor.

In some embodiments, the binder comprises carboxymethylcellulose; and/or the mold inhibitor comprises calcium propionate.

In some embodiments, the shrimp feed comprises: proteins, amino acids, carbohydrates and starches, vitamins and minerals, fatty acids and phospholipids, and non-nutrient additives.

In some embodiments, the: proteins and amino acids comprise: from 20% to 65% wt/v of the composition; carbohydrates and starches comprise: from 15% to 40% wt/v of the composition; vitamins and minerals comprise: from 0% to 10% wt/v of the composition; fatty acids and phospholipids comprise: from 0% to 10% wt/v of the composition; and non-nutrient additives comprise: from 0% to 10% wt/v of the composition.

In some embodiments, the shrimp feed comprises: anchovy meal, soybean meal, fish oil, tuna oil, wheat flour, krill meal, wheat gluten meal, choline chloride with choline, cholesterol, soy lecithin, Ca(H₂PO₄)₂, salt, Rovimix-stay-C 35, shrimp pre-mix, methionine, threonine, lysine, carboxymethylcellulose, and calcium propionate.

In some embodiments, the: anchovy meal comprises about 26.5% wt/v of the shrimp feed; soybean meal comprises about 30.4% wt/v of the shrimp feed: fish oil and tuna oil comprise about 1.8% wt/v of the shrimp feed: wheat flour comprises about 27.27% wt/v of the shrimp feed; krill meal comprises about 2% wt/v of the shrimp feed; wheat gluten meal comprises about 3.5% wt/v of the shrimp feed; choline chloride with choline comprises about 1% wt/v of the shrimp feed; cholesterol comprises about 0.03% wt/v of the shrimp feed; soy lecithin comprises about 1.5% wt/v of the shrimp feed; Ca(H₂PO₄)₂ comprises about 2% wt/v of the shrimp feed; salt comprises about 0.6% wt/v of the shrimp feed; Rovimix-stay-C 35 comprises about 0.1% wt/v of the shrimp feed; shrimp pre-mix comprises about 0.4% wt/v of the shrimp feed; methionine comprises about 0.2% wt/v of the shrimp feed; threonine comprises about 0.3% wt/v of the shrimp feed: lysine comprises about 0.3% wt/v of the shrimp feed; carboxymethylcellulose comprises about 2% N wt/v of the shrimp feed; and calcium propionate comprises about 0.10% wt/v of the shrimp feed.

In some embodiments, the bacteriocin comprises from about 0.0001% to 20% wt/v of the composition.

In some embodiments, the bacteriocin comprises from about 1×10⁻³ mg/g to 200 mg/g of the composition.

In some embodiments, the Nisin comprises from about 0.0001% to 10% wt/v of the composition.

In some embodiments, the: shrimp feed comprises about 99.99% wt/v of the composition; and the bacteriocin comprises about 0.010% w/v of the composition.

In some embodiments, the Nisin comprises Nisin A, Nisin E, Nisin F, Nisin G, Nisin H, Nisin J, Nisin O, Nisin P, Nisin Q, Nisin U, Nisin U2, Nisin P, Nisin Z, combinations thereof, and engineered variants thereof. In some embodiments, any suitable Nisin, now known or later discovered, is used. In some embodiments, the Nisin comprises Nisin Z.

In some embodiments, the composition further comprises ethylenediaminetetraacetic acid (EDTA).

In some embodiments, the EDTA comprises from about 0.0001% to 2% wt/v of the composition.

In some embodiments, the EDTA comprises from about 0.001 mg/g to 20 mg/g of the composition.

In some embodiments, the shrimp feed comprises about 99.98% wt/v of the composition: the bacteriocin comprises about 0.01% w/v of the composition; and the EDTA comprises about 0.01% wt/v of the composition.

The present disclosure also provides compositions comprising: a bacteriocin, wherein the bacteriocin comprises Nisin Z, and wherein the bacteriocin comprises about 0.01% w/v of the composition; and shrimp feed, wherein the shrimp feed comprises about 99.99% wt/v of the composition, and wherein: anchovy meal comprises about 26.5% wt/v of the shrimp feed; soybean meal comprises about 30.4% wt/v of the shrimp feed; fish oil and tuna oil comprise about 1.8% wt/v of the shrimp feed; wheat flour comprises about 27.27% wt/v of the shrimp feed; krill meal comprises about 2% wt/v of the shrimp feed; wheat gluten meal comprises about 3.5% wt/v of the shrimp feed; choline chloride with choline comprises about 10% wt/v of the shrimp feed; cholesterol comprises about 0.03% wt/v of the shrimp feed; soy lecithin comprises about 1.5% wt/v of the shrimp feed; Ca(H₂PO₄)₂ comprises about 2% wt/v of the shrimp feed; salt comprises about 0.6% N wt/v of the shrimp feed: Rovimix-stay-C 35 comprises about 0.1% wt/v of the shrimp feed; shrimp pre-mix comprises about 0.4% wt/v of the shrimp feed; methionine comprises about 0.2% wt/v of the shrimp feed; threonine comprises about 0.3% wt/v of the shrimp feed; lysine comprises about 0.3% wt/v of the shrimp feed; carboxymethylcellulose comprises about 2% wt/v of the shrimp feed; and calcium propionate comprises about 0.1% wt/v of the shrimp feed, wherein the composition prevents or treats a disease or one or more symptoms thereof caused by pathogenic Vibrio in a shrimp in need thereof.

The present disclosure also provides compositions comprising: a bacteriocin, wherein the bacteriocin comprises Nisin Z, and wherein the bacteriocin comprises about 0.010% w/v of the composition; EDTA, wherein the EDTA comprises about 0.010% wt/v of the composition; and shrimp feed, wherein the shrimp feed comprises about 99.98% wt/v of the composition, and wherein: anchovy meal comprises about 26.5% wt/v of the shrimp feed; soybean meal comprises about 30.4% wt/v of the shrimp feed; fish oil and tuna oil comprise about 1.8% wt/v of the shrimp feed; wheat flour comprises about 27.27% wt/v of the shrimp feed; krill meal comprises about 2% wt/v of the shrimp feed; wheat gluten meal comprises about 3.5% wt/v of the shrimp feed; choline chloride with choline comprises about 1% wt/v of the shrimp feed: cholesterol comprises about 0.03% wt/v of the shrimp feed; soy lecithin comprises about 1.5% wt/v of the shrimp feed; Ca(H₂PO₄)₂ comprises about 2% wt/v of the shrimp feed; salt comprises about 0.6% wt/v of the shrimp feed; Rovimix-stay-C 35 comprises about 0.10% wt/v of the shrimp feed; shrimp pre-mix comprises about 0.4% wt/v of the shrimp feed; methionine comprises about 0.2% wt/v of the shrimp feed; threonine comprises about 0.3% wt/v of the shrimp feed: lysine comprises about 0.3% wt/v of the shrimp feed; carboxymethylcellulose comprises about 2% wt/v of the shrimp feed; and calcium propionate comprises about 0.10% wt/v of the shrimp feed, wherein the composition prevents or treats a disease or one or more symptoms thereof caused by pathogenic Vibrio in a shrimp in need thereof.

In some embodiments, the bacteriocin further comprises: a Class I, Class II, and/or Class III bacteriocin.

In some embodiments, the Class I, Class II, and/or Class III bacteriocin comprises a colicin, colicin-like bacteriocin, klebicin, microcin, or phage-tail-like bacteriocin.

In some embodiments, the bacteriocin and shrimp feed are combined.

In some embodiments, combined comprises: top-coating, extrusion, or mixing-in.

In some embodiments, the composition is administered to the shrimp in need thereof.

In some embodiments, the shrimp in need thereof is or has not been administered an antibiotic.

In some embodiments, upon administration, a concentration of pathogenic Vibrio is reduced in the shrimp.

In some embodiments, upon administration, the one or more symptoms thereof is reduced in the shrimp.

In some embodiments, upon administration, one or more diseases caused by pathogenic Vibrio or symptoms thereof in the shrimp are reduced, prevented, or treated.

In some embodiments, the one or more symptoms comprise lethargy, anorexia, slow growth, an empty digestive tract, pale-to-white hepatopancreases, reddish body discoloration, yellowing gill tissue, appearance of white patches in abdominal muscle, melanisation, granulomatous encapsulation, luminescence, a soft shell, melanised spots or streaks visible within the hepatopancreas, and atrophy of the hepatopancreas.

In some embodiments, upon administration, a microbiome of the shrimp is modified relative to a suitable control.

In some embodiments, the pathogenic Vibrio comprise Vibrio parahaemolyticus, Vibrio alginolyticus, Vibrio vulnificus, Vibrio cholera, Vibrio harveyi, Vibrio fischeri, Vibrio anguillarum, Vibrio spledidus, Vibrio nerves, Vibrio tubiashii, Vibrio campbellii, Vibrio aestuarianus, Vibrio pelagius, Vibrio mediterranei, Vibrio diazotrophicus, Vibrio damselae, Vibrio mimicus, and Vibrio cholera.

In some embodiments, the disease comprises: acute hepatopancreatic necrosis disease (AHPND), vibriosis, loose shell syndrome (LSS), tail necrosis, shell disease, red disease, white gut disease (WGD), white tail disease, white feces disease (WFD), luminous bacterial disease, and necrotizing hepatopancreatitis.

In some embodiments, the composition does not comprises an antibiotic.

In some embodiments, the shrimp in need thereof is a farmed shrimp.

The present disclosure also provides methods of reducing a concentration of pathogenic Vibrio in a shrimp in need thereof, the method comprising administering a composition comprising: a bacteriocin, wherein the bacteriocin comprises Nisin Z, and wherein the bacteriocin comprises about 0.01% w/v of the composition; and shrimp feed, wherein the shrimp feed comprises about 99.99% wt/v of the composition, and wherein: anchovy meal comprises about 26.5% wt/v of the shrimp feed; soybean meal comprises about 30.4% wt/v of the shrimp feed; fish oil and tuna oil comprise about 1.8% wt/v of the shrimp feed; wheat flour comprises about 27.27% wt/v of the shrimp feed; krill meal comprises about 2% wt/v of the shrimp feed; wheat gluten meal comprises about 3.5% wt/v of the shrimp feed: choline chloride with choline comprises about 1% wt/v of the shrimp feed: cholesterol comprises about 0.03% wt/v of the shrimp feed: soy lecithin comprises about 1.5% wt/v of the shrimp feed; Ca(H₂PO₄)₂ comprises about 2% wt/v of the shrimp feed; salt comprises about 0.6% wt/v of the shrimp feed; Rovimix-stay-C 35 comprises about 0.1% wt/v of the shrimp feed; shrimp pre-mix comprises about 0.4% wt/v of the shrimp feed; methionine comprises about 0.2% wt/v of the shrimp feed; threonine comprises about 0.3% wt/v of the shrimp feed; lysine comprises about 0.3% wt/v of the shrimp feed; carboxymethylcellulose comprises about 2% wt/v of the shrimp feed; and calcium propionate comprises about 0.10% wt/v of the shrimp feed.

The present disclosure also provides methods of preventing or treating a disease or one or more symptoms thereof caused by pathogenic Vibrio in a shrimp in need thereof, the method comprising administering a composition comprising: a bacteriocin, wherein the bacteriocin comprises Nisin Z. and wherein the bacteriocin comprises about 0.01% w/v of the composition; and shrimp feed, wherein the shrimp feed comprises about 99.99% wt/v of the composition, and wherein: anchovy meal comprises about 26.5% wt/v of the shrimp feed; soybean meal comprises about 30.4% wt/v of the shrimp feed; fish oil and tuna oil comprise about 1.8% wt/v of the shrimp feed: wheat flour comprises about 27.27% wt/v of the shrimp feed; krill meal comprises about 2% wt/v of the shrimp feed: wheat gluten meal comprises about 3.5% wt/v of the shrimp feed; choline chloride with choline comprises about 1% v wt/v of the shrimp feed; cholesterol comprises about 0.03% wt/v of the shrimp feed; soy lecithin comprises about 1.5% wt/v of the shrimp feed; Ca(H₂PO₄)₂ comprises about 2% wt/v of the shrimp feed; salt comprises about 0.6% wt/v of the shrimp feed; Rovimix-stay-C 35 comprises about 0.1% wt/v of the shrimp feed; shrimp pre-mix comprises about 0.4% wt/v of the shrimp feed; methionine comprises about 0.2% wt/v of the shrimp feed; threonine comprises about 0.3% wt/v of the shrimp feed; lysine comprises about 0.3% wt/v of the shrimp feed; carboxymethylcellulose comprises about 2% wt/v of the shrimp feed; and calcium propionate comprises about 0.1% wt/v of the shrimp feed.

The present disclosure also provides methods of reducing a concentration of pathogenic Vibrio in a shrimp in need thereof, the method comprising administering a composition comprising: a bacteriocin, wherein the bacteriocin comprises Nisin Z. and wherein the bacteriocin comprises about 0.01% w/v of the composition; EDTA, wherein the EDTA comprises about 0.010% wt/v of the composition; and shrimp feed, wherein the shrimp feed comprises about 99.98% wt/v of the composition, and wherein: anchovy meal comprises about 26.5% wt/v of the shrimp feed: soybean meal comprises about 30.4% wt/v of the shrimp feed: fish oil and tuna oil comprise about 1.8% wt/v of the shrimp feed; wheat flour comprises about 27.27% wt/v of the shrimp feed; krill meal comprises about 2% wt/v of the shrimp feed; wheat gluten meal comprises about 3.5% wt/v of the shrimp feed; choline chloride with choline comprises about 1% wt/v of the shrimp feed; cholesterol comprises about 0.03% wt/v of the shrimp feed: soy lecithin comprises about 1.5% wt/v of the shrimp feed; Ca(H₂PO₄)₂ comprises about 2% wt/v of the shrimp feed; salt comprises about 0.6% wt/v of the shrimp feed; Rovimix-stay-C 35 comprises about 0.10% wt/v of the shrimp feed; shrimp pre-mix comprises about 0.4% wt/v of the shrimp feed; methionine comprises about 0.2% wt/v of the shrimp feed; threonine comprises about 0.3% wt/v of the shrimp feed; lysine comprises about 0.3% wt/v of the shrimp feed; carboxymethylcellulose comprises about 2% wt/v of the shrimp feed; and calcium propionate comprises about 0.10% wt/v of the shrimp feed.

The present disclosure also provides methods of preventing or treating a disease or one or more symptoms thereof caused by pathogenic Vibrio in a shrimp in need thereof, the method comprising administering a composition comprising: a bacteriocin, wherein the bacteriocin comprises Nisin Z. and wherein the bacteriocin comprises about 0.01% w/v of the composition; EDTA, wherein the EDTA comprises about 0.01% wt/v of the composition; and shrimp feed, wherein the shrimp feed comprises about 99.98% wt/v of the composition, and wherein: anchovy meal comprises about 26.5% wt/v of the shrimp feed; soybean meal comprises about 30.4% wt/v of the shrimp feed; fish oil and tuna oil comprise about 1.8% wt/v of the shrimp feed: wheat flour comprises about 27.27% wt/v of the shrimp feed; krill meal comprises about 2% wt/v of the shrimp feed; wheat gluten meal comprises about 3.5% wt/v of the shrimp feed; choline chloride with choline comprises about 1% wt/v of the shrimp feed; cholesterol comprises about 0.03% wt/v of the shrimp feed; soy lecithin comprises about 1.5% wt/v of the shrimp feed; Ca(H₂PO₄)₂ comprises about 2% wt/v of the shrimp feed; salt comprises about 0.6% wt/v of the shrimp feed; Rovimix-stay-C 35 comprises about 0.10% wt/v of the shrimp feed; shrimp pre-mix comprises about 0.4% wt/v of the shrimp feed; methionine comprises about 0.2% wt/v of the shrimp feed; threonine comprises about 0.3% wt/v of the shrimp feed; lysine comprises about 0.3% wt/v of the shrimp feed; carboxymethylcellulose comprises about 2% wt/v of the shrimp feed; and calcium propionate comprises about 0.10% wt/v of the shrimp feed.

### BRIEF DESCRIPTION OF THE DRAWINGS

FIG. 1 is a graph plotting the cumulative survival of shrimp over time when the shrimp's diet consisted of a composition of the disclosure, including a bacteriocin, Nisin (e.g., Nisin A and Nisin Z), and shrimp feed, as compared to negative and positive controls. Three experimental conditions were tested, including shrimp feed having 84 mg/g Nisin, 8.4 mg/g Nisin, and 0.84 mg/g Nisin.

FIGS. 2A-2E are a set of graphs plotting the same data of cumulative survival of shrimp described in FIG. 1, from top-left-to-top-right and top-to-bottom including the 84/g Nisin group, the 8.4 mg/g Nisin group, the 0.84 mg/g Nisin group, the negative control group, and the positive control group, respectively.

FIG. 3 is a graph plotting the percent mortality of shrimp as a function of administered Nisin A (10% pure) at 24 hours and 48 hours post-administration.

FIG. 4 is a graph plotting the percent mortality of shrimp as a function of administered Nisin Z (26.7% pure) at 24 hours and 48 hours post-administration.

FIG. 5 is a graph plotting the percent mortality of shrimp as a function of administered ethylenediaminetetraacetic acid (EDTA) concentration at 24 hours and 48 hours post-administration.

FIG. 6 is a graph plotting the zones of inhibition (ZOI) diameter in Staphylococcus aureus (a control strain), two strains of E. coli (“ECY,” an experimental strain with low sensitivity to Nisin, and “ECX,” an experimental strain with high sensitivity to Nisin), and two strains of Vibrio parahaemolyticus (“VPY,” an experimental strain with low sensitivity to Nisin and “VPX,” an experimental strain with high sensitivity to Nisin) administered Nisin (high purity 96%); or Nisin (high purity 96%) with EDTA.

FIG. 7 is a graph plotting the zones of inhibition (ZOI) diameter in Staphylococcus aureus (a control strain), two strains of E. coli (“ECY,” an experimental strain with low sensitivity to Nisin, and “ECX, an experimental strain with high sensitivity to Nisin), and two strains of Vibrio parahaemolyticus (“VPY,” an experimental strain with low sensitivity to Nisin and “VPX,” an experimental strain with high sensitivity to Nisin) administered Nisin (low purity 10%); or Nisin (low purity 10%) with EDTA.

### DETAILED DESCRIPTION

### Definitions

As used in the specification and the appended claims, the singular forms “a,” “an” and “the” include plural referents unless the context clearly dictates otherwise.

As used herein, the expression “and/or” in connection with two or more recited objects comprises individually each of the recited objects and the various combinations of two or more of the recited objects, unless otherwise understood from the context and use. As used herein, unless specifically indicated otherwise, the word “or” is used in the inclusive sense of “and/or” and not the exclusive sense of “either/or.”

The use of the term “include,” “comprises,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the terms “about” or “substantially” is before a quantitative value, the present disclosure also comprises the specific quantitative value itself, unless specifically stated otherwise. As used herein, the term “about” refers to a 10% variation from the nominal value unless otherwise indicated or inferred.

Throughout the description, where compositions are described as having, including, or including specific components, or where processes and methods are described as having, including, or including specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a method described herein can be combined in a variety of ways without departing from the spirit and scope of the present invention, whether explicit or implicit herein. For example, where reference is made to a particular compound, that compound can be used in various embodiments of compositions disclosed herein and/or in methods disclosed herein, unless otherwise understood from the context. In other words, within this application, embodiments have been described and depicted in a way that enables a clear and concise application to be written and drawn, but it is intended and will be appreciated that embodiments may be variously combined or separated without parting from the present teachings. For example, it will be appreciated that all features described and depicted herein can be applicable to all aspects of the invention(s) described and depicted herein.

Moreover, any feature or combination of features set forth herein can be excluded or omitted.

It should be understood that the expression “at least one of” comprises individually each of the recited objects after the expression and the various combinations of two or more of the recited objects unless otherwise understood from the context and use.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Bacteria produce numerous antimicrobial substances including conventional small molecule antibiotics, ribosomally-synthesized peptides and proteins known as bacteriocins and microcins, bacteriocin-like inhibitory substances, as well as organic acids, hydrogen peroxide, and diacetyl. Many of these inhibitory antimicrobial substances have narrow inhibitory spectra. Some of these inhibitory substances are produced in response to signals received from a potential competitor which then elicits an antagonistic response. Growth inhibition of one isolate by another may be due to the production of a range of antimicrobial substances, including conventional small molecule antibiotics, ribosomally-synthesized peptides and proteins known as bacteriocins and microcins, bacteriocin-like inhibitory substances, as well as organic acids, hydrogen peroxide, and diacetyl.

The term “bacteriocin” as used herein refer a class of proteinaceous toxins produced by bacteria to inhibit the growth of similar or closely related bacterial strain(s). Bacteriocins are an example of inducible antimicrobials. Bacteriocins are a frequently encountered family of bacterial antimicrobials. Bacteriocins are ribosomally-synthesized antimicrobial peptides and proteins produced by many species of bacteria, which often preferentially target close relatives of the producing species. Several studies have suggested that virtually all bacteria are able to produce bacteriocins. Bacteriocins possess characteristics that make them particularly appealing for consideration as an alternative to antibiotics. Bacteriocins exhibit little to no toxicity to humans, with several identified generally recognized as safe (GRAS) by the FDA and used in food preservation for over fifty years. Exemplary bacteriocins of the disclosure include, for example, but are not limited to a Class I, Class II, and Class III bacteriocin. For example, the Class I, Class II, and Class III bacteriocins include, for example, a colicin, colicin-like bacteriocin, klebicin, microcin, or phage-tail-like bacteriocin.

### COMPOUNDS OF THE DISCLOSURE

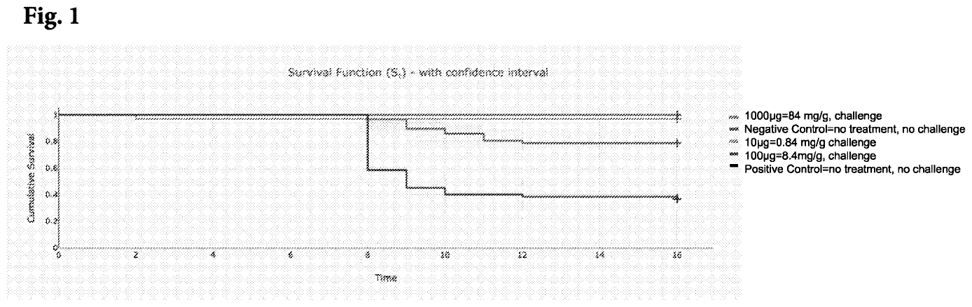

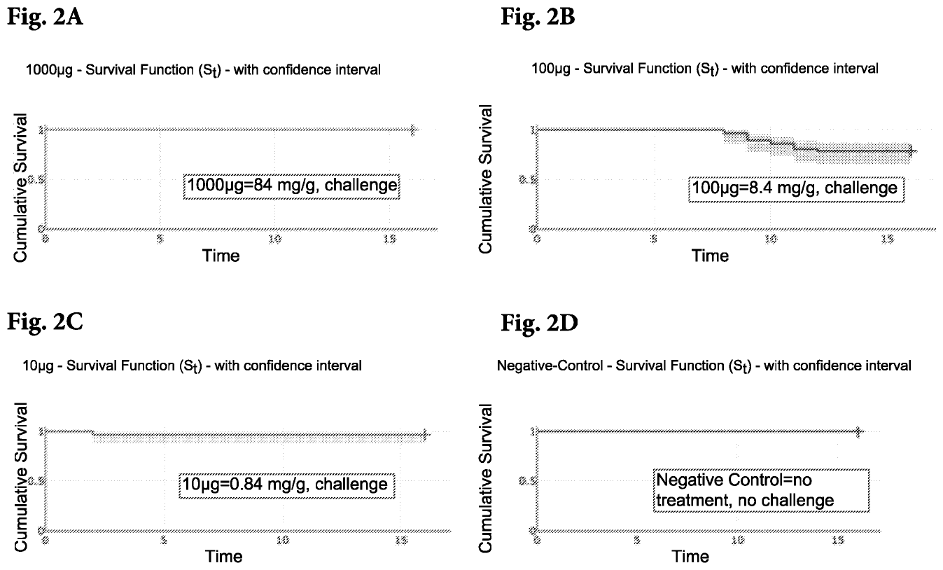

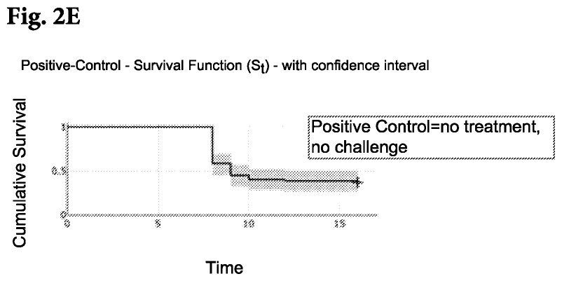

The present disclosure is based, in part, upon the surprising discovery that the compositions herein, including a bacteriocin (e.g., acidocin, actagardine, agrocin, alveicin, aureocin, aureocin A53, aureocin A70, bisin, carnocin, camocyclin, caseicin, cerein, circularin A, colicin, curvaticin, divercin, duramycin, enterocin, enterolysin, epidermin/gallidermin, erwiniocin, gardimycin, gassericin A, glycinecin, halocin, haloduracin, klebicin, lactocin S, lactococcin, lacticin, leucoccin, lysostaphin, macedocin, mersacidin, mesentericin, microbisporicin, microcin, microcin S, mutacin, Nisin (e.g., Nisin A and Nisin Z), paenibacillin, planosporicin, pediocin, pentocin, plantaricin, pneumocyclicin, pyocin, reutericin 6, reutericyclin, reuterin, sakacin, salivaricin, sublancin, subtilin, sulfolobicin, tasmancin, thuricin 17, trifolitoxin, variacin, vibriocin, wamericin, or wamerin) and shrimp feed significantly reduce the concentration of pathogenic Vibrio (See Example 1) and significantly increase the survival of shrimp in need thereof from Vibrio-associated disease (e.g., FIG. 1 and FIGS. 2A-2E). For example, disclosed herein is a composition including: a bacteriocin; and shrimp feed, wherein the composition prevents or treats a disease or one or more symptoms thereof caused by pathogenic Vibrio in a shrimp in need thereof, and wherein the bacteriocin comprises Nisin (e.g., Nisin A, Nisin E, Nisin F, Nisin G, Nisin H, Nisin J, Nisin O, Nisin P, Nisin Q, Nisin U, Nisin U2, Nisin P, Nisin Z, combinations thereof, and engineered variants thereof).

In some embodiments, the bacteriocin is Nisin (e.g., Nisin A, Nisin E, Nisin F, Nisin G, Nisin H, Nisin J, Nisin O, Nisin P, Nisin Q, Nisin U, Nisin U2, Nisin P, Nisin Z, combinations thereof, and engineered variants thereof). In some embodiments, the Nisin is Nisin A. In some embodiments, the Nisin is Nisin E. In some embodiments, the Nisin is Nisin F. In some embodiments, the Nisin is Nisin G. In some embodiments, the Nisin is Nisin H. In some embodiments, the Nisin is Nisin J. In some embodiments, the Nisin is Nisin O. In some embodiments, the Nisin is Nisin P. In some embodiments, the Nisin is Nisin Q. In some embodiments, the Nisin is Nisin U. In some embodiments, the Nisin is Nisin U2. In some embodiments, the Nisin is Nisin P. In some embodiments, the Nisin is Nisin Z. In some embodiments, the Nisin is a combination of Nisin A, Nisin E, Nisin F, Nisin G, Nisin H, Nisin J, Nisin O, Nisin P, Nisin Q, Nisin U, Nisin U2, Nisin P, Nisin Z, and engineered variants thereof. In some embodiments, any suitable Nisin, now known or later discovered, is used.

In some embodiments, the Nisin is Nisin Z.

In some embodiments, the bacteriocin further comprises: a Class I, Class II, and/or Class III bacteriocin.

Class I bacteriocins are small peptide inhibitors and include Nisin and other lantibiotics.

The class II bacteriocins are small (<10 kDa) heat-stable proteins. This class is subdivided into five subclasses. The class IIa bacteriocins (pediocin-like bacteriocins) are the largest subgroup and contain an N-terminal consensus sequence -Tyr-Gly-Asn-Gly-Val-Xaa-Cys across this group. The class IIb bacteriocins (two-peptide bacteriocins) require two different peptides for activity. One such an example is lactococcin G, which permeabilizes cell membranes for monovalent sodium and potassium cations, but not for divalent cations. Almost all of these bacteriocins have a GxxxG motifs. This motif is also found in transmembrane proteins, where they are involved in helix-helix interactions. Class IIc encompasses cyclic peptides, in which the N-terminal and C-terminal regions are covalentely linked. Enterocin AS-48 is the prototype of this group. Class IId cover single-peptide bacteriocins, which are not post-translationally modified and do not show the pediocin-like signature. An example of this group is the highly stable aureocin A53. This bacteriocin is stable under highly acidic conditions, high temperatures, and is not affected by proteases. The most recently proposed subclass is the Class IIe, which encompasses those bacteriocins composed of three or four non-pediocin like peptides. An example is aureocin A70, a four-peptide bacteriocin, highly active against Listeria monocytogenes.

Class III bacteriocins are large, heat-labile (>10 kDa) protein bacteriocins. This class is subdivided in two subclasses: subclass IIIa (bacteriolysins) and subclass IIIb. Subclass IIIa comprises those peptides that kill bacterial cells by cell wall degradation, thus causing cell lysis. Subclass IIIb, in contrast, comprises those peptides that do not cause cell lysis, killing the target cells by disrupting plasma membrane potential.

For example, in some embodiments, the bacteriocin further comprises a Class I bacteriocin. In some embodiments, the bacteriocin further comprises a Class II bacteriocin. In some embodiments, the bacteriocin further comprises a Class III bacteriocin.

In some embodiments, Class I, Class II, and/or Class III bacteriocin comprises a colicin, colicin-like bacteriocin, klebicin, microcin, or phage-tail-like bacteriocin. For example, in some embodiments, the bacteriocin further comprises a colicin. In some embodiments, the bacteriocin further comprises a colicin-like bacteriocin. In some embodiments, the bacteriocin further comprises a klebicin. In some embodiments, the bacteriocin further comprises a microcin. In some embodiments, the bacteriocin further comprises a phage-tail-like bacteriocin.

Other exemplary, non-limiting examples of Class I, II, and III bacteriocins include acidocin, actagardine, agrocin, alveicin, aureocin, aureocin A53, aureocin A70, bisin, carnocin, carnocyclin, caseicin, cerein, circularin A, colicin, curvaticin, divercin, duramycin, enterocin, enterolysin, epidermin/gallidermin, erwiniocin, gardimycin, gassericin A, glycinecin, halocin, haloduracin, klebicin, lactocin S, lactococcin, lacticin, leucoccin, lysostaphin, macedocin, mersacidin, mesentericin, microbisporicin, microcin, microcin S, mutacin, Nisin, paenibacillin, planosporicin, pediocin, pentocin, plantaricin, pneumocyclicin, pyocin, reutericin 6, reutericyclin, reuterin, sakacin, salivaricin, sublancin, subtilin, sulfolobicin, tasmancin, thuricin 17, trifolitoxin, variacin, vibriocin, wamericin, and wamerin.

In some embodiments, the shrimp feed comprises: one or more proteins (e.g., fish meal, soybean meal, shrimp meal, sardine meal, poultry meal, squid meal, corn gluten meal, krill meal, insect meal, and proteins from a single-cell source), amino acids (e.g., methionine, threonine, lysine, arginine, valine, histidine, phenylalanine, and tryptophan), carbohydrates and/or starches (e.g., wheat flour, wheat gluten meal, corn, and rice), vitamins and/or minerals (e.g., pre-mix, Ca(H₂PO₄)₂ with vitamin C, choline chlorine with chloride, salt, potassium chloride, calcium carbonate, magnesium sulfate, and monosodium phosphate), fatty acids and/or phospholipids (e.g., fish oil, tuna oil, squid oil, krill oil, algae oil, soy lecithin oil, and vegetable oil), non-nutrient additives (e.g., a binder (e.g. carboxymethylcellulose), soy lecithin, and a mold inhibitor (e.g., calcium propionate)), and combinations thereof. For example, in some embodiments, the shrimp feed comprises one or more proteins. In some embodiments, the shrimp feed comprises one or more amino acids. In some embodiments, the shrimp feed comprises one or more carbohydrates and/or starches. In some embodiments, the shrimp feed comprises one or more vitamins and/or minerals. In some embodiments, the shrimp feed comprises one or more fatty acids and/or phospholipids. In some embodiments, the shrimp feed comprises one or more non-nutrient additives.

In some embodiments, the shrimp feed comprises one or more proteins, amino acids, carbohydrates and starches, vitamins and minerals, fatty acids and phospholipids, and non-nutrient additives.

In some embodiments, the shrimp feed comprises one or more proteins. For example, in some embodiments, the one or more proteins include: fish meal, soybean meal, shrimp meal, sardine meal, poultry meal, squid meal, corn gluten meal, krill meal, insect meal, and proteins from a single-cell source. In some embodiments, the one or more proteins is fish meal. In some embodiments, the one or more proteins is soybean meal. In some embodiments, the one or more proteins is shrimp meal. In some embodiments, the one or more proteins is sardine meal. In some embodiments, the one or more proteins is poultry meal. In some embodiments, the one or more proteins is squid meal. In some embodiments, the one or more proteins is corn gluten meal. In some embodiments, the one or more proteins is krill meal. In some embodiments, the one or more proteins is insect meal. In some embodiments, the one or more proteins is a protein from a single-cell source (e.g., such a protein from a single-cell source can be from a product manufacturer, such as Calysta and KnipBio).

In some embodiments, the shrimp feed comprises one or more amino acids. For example, in some embodiments, the one or more amino acids include: methionine, threonine, lysine, arginine, valine, histidine, phenylalanine, and tryptophan. In some embodiments, the one or more amino acids is methionine. In some embodiments, the one or more amino acids is threonine. In some embodiments, the one or more amino acids is lysine. In some embodiments, the one or more amino acids is arginine. In some embodiments, the one or more amino acids is valine. In some embodiments, the one or more amino acids is histidine. In some embodiments, the one or more amino acids is phenylalanine. In some embodiments, the one or more amino acids is tryptophan.

In some embodiments, the shrimp feed comprises one or more carbohydrates and/or starches. For example, in some embodiments, the one or more carbohydrates and/or starches include: wheat flour, wheat gluten meal, corn, and rice. In some embodiments, the one or more carbohydrates and/or starches is wheat flour. In some embodiments, the one or more carbohydrates and/or starches is wheat gluten meal. In some embodiments, the one or more carbohydrates and/or starches is corn. In some embodiments, the one or more carbohydrates and/or starches is rice.

In some embodiments, the shrimp feed comprises one or more vitamins and/or minerals. For example, in some embodiments, the one or more vitamins and/or minerals include: pre-mix. Ca(H₂PO₄)₂ with vitamin C, choline chlorine with chloride, salt, potassium chloride, calcium carbonate, magnesium sulfate, and monosodium phosphate. In some embodiments, the one or more vitamins and/or minerals is pre-mix. In some embodiments, the one or more vitamins and/or minerals is Ca(H₂PO₄)₂ with vitamin C. In some embodiments, the one or more vitamins and/or minerals is choline chlorine with chloride. In some embodiments, the one or more vitamins and/or minerals is salt. In some embodiments, the one or more vitamins and/or minerals is potassium chloride. In some embodiments, the one or more vitamins and/or minerals is calcium carbonate. In some embodiments, the one or more vitamins and/or minerals is magnesium sulfate. In some embodiments, the one or more vitamins and/or minerals is monosodium phosphate.

In some embodiments, the shrimp feed comprises one or more fatty acids and/or phospholipids. For example, in some embodiments, the one or more fatty acids and/or phospholipids include: fish oil, tuna oil, squid oil, krill oil, algae oil, soy lecithin oil, and vegetable oil. In some embodiments, the one or more fatty acids and/or phospholipids is fish oil. In some embodiments, the one or more fatty acids and/or phospholipids is tuna oil. In some embodiments, the one or more fatty acids and/or phospholipids is squid oil. In some embodiments, the one or more fatty acids and/or phospholipids is krill oil. In some embodiments, the one or more fatty acids and/or phospholipids is algae oil. In some embodiments, the one or more fatty acids and/or phospholipids is soy lecithin oil. In some embodiments, the one or more fatty acids and/or phospholipids is vegetable oil.

In some embodiments, the shrimp feed comprises one or more non-nutrient additives. For example, in some embodiments, the one or more non-nutrient additives include: a binder (e.g., carboxymethylcellulose), soy lecithin, and a mold inhibitor (e.g., calcium propionate). In some embodiments, the one or more non-nutrient additives is a binder. In some embodiments, the binder is carboxymethylcellulose. In some embodiments, the one or more non-nutrient additives is soy lecithin. In some embodiments, the one or more non-nutrient additives is a mold inhibitor. In some embodiments, the mold inhibitor is calcium propionate.

In some embodiments, any suitable distribution of the proteins, amino acids, carbohydrates and starches, vitamins and minerals, fatty acids and phospholipids, and/or non-nutrient additives is combined to make the shrimp feed. For example, in some embodiments, the one or more: (a) proteins (e.g., fish meal, soybean meal, shrimp meal, sardine meal, poultry meal, squid meal, corn gluten meal, krill meal, insect meal, and proteins from a single-cell source) and amino acids (e.g., methionine, threonine, lysine, arginine, valine, histidine, phenylalanine, and tryptophan) include: from 20% to 65% wt/v of the composition: (b) carbohydrates and starches (e.g., wheat flour, wheat gluten meal, corn, and rice) include: from 15% to 40% wt/v of the composition; (c) vitamins and minerals (e.g., pre-mix, Ca(H₂PO₄)₂ with vitamin C, choline chlorine with chloride, salt, potassium chloride, calcium carbonate, magnesium sulfate, and monosodium phosphate) include: from 0% to 10% wt/v of the composition: (d) fatty acids and phospholipids (e.g., fish oil, tuna oil, squid oil, krill oil, algae oil, soy lecithin oil, and vegetable oil) include: from 0% to 10% wt/v of the composition; and (e) non-nutrient additives (e.g., a binder (e.g., carboxymethylcellulose), soy lecithin, and a mold inhibitor (e.g., calcium propionate)) include: from 0% to 10% wt/v of the composition. In some embodiments, the Nisin comprises from about 0.0001% to 10% wt/v of the composition.

For example, in some embodiments, the one or more proteins (e.g., fish meal, soybean meal, shrimp meal, sardine meal, poultry meal, squid meal, corn gluten meal, krill meal, insect meal, and proteins from a single-cell source) and amino acids (e.g., methionine, threonine, lysine, arginine, valine, histidine, phenylalanine, and tryptophan) include: from 20% to 65% (e.g., 25% to 60%, 30% to 50%, or about 40%) wt/v of the composition. In some embodiments, the one or more proteins and amino acids include from 25% to 60% wt/v of the composition. In some embodiments, the one or more proteins and amino acids include from 30% to 50% wt/v of the composition. In some embodiments, the one or more proteins and amino acids comprises about 40% wt/v of the composition.

In some embodiments, the one or more proteins (e.g., fish meal, soybean meal, shrimp meal, sardine meal, poultry meal, squid meal, corn gluten meal, krill meal, insect meal, and proteins from a single-cell source) and amino acids (e.g. methionine, threonine, lysine, arginine, valine, histidine, phenylalanine, and tryptophan) include: less than 65% (e.g., 60%, 55%, 50%, 40%, 30%, and 20%) wt/v of the composition. In some embodiments, one or more proteins and amino acids comprises less than 60% of the composition. In some embodiments, one or more proteins and amino acids comprises less than 55% wt/v of the composition. In some embodiments, one or more proteins and amino acids comprises less than 50% wt/v of the composition. In some embodiments, one or more proteins and amino acids comprises less than 40% wt/v of the composition. In some embodiments, one or more proteins and amino acids comprises less than 30% wt/v of the composition. In some embodiments, one or more proteins and amino acids comprises less than 20% wt/v of the composition.

In some embodiments, the one or more carbohydrates and starches (e.g., wheat flour, wheat gluten meal, corn, and rice) include: from 15% to 40% (e.g., 20% to 35%, or about 30%) wt/v of the composition. In some embodiments, the one or more carbohydrates and starches include from 20% to 35% wt/v of the composition. In some embodiments, the one or more carbohydrates and starches comprises about 30% wt/v of the composition.

In some embodiments, the one or more carbohydrates and starches (e.g., wheat flour, wheat gluten meal, corn, and rice) include: less than 40% (e.g., 35%, 30%, 20% or 15%) wt/v of the composition. In some embodiments, the one or more carbohydrates and starches include: less than 35% wt/v of the composition. In some embodiments, the one or more carbohydrates and starches include: less than 30% wt/v of the composition. In some embodiments, the one or more carbohydrates and starches include: less than 20% wt/v of the composition. In some embodiments, the one or more carbohydrates and starches include: less than 15% wt/v of the composition.

In some embodiments, the one or more vitamins and minerals (e.g., pre-mix, Ca(H₂PO₄)₂ with vitamin C, choline chlorine with chloride, salt, potassium chloride, calcium carbonate, magnesium sulfate, and monosodium phosphate) include: from 0% to 10% (e.g., about 5%) wt/v of the composition. In some embodiments, the more vitamins and minerals comprises about 5% w/v of the composition.

In some embodiments, the one or more vitamins and minerals (e.g., pre-mix, Ca(H₂PO₄)₂ with vitamin C, choline chlorine with chloride, salt, potassium chloride, calcium carbonate, magnesium sulfate, and monosodium phosphate) include: less than 10% (e.g., 9%, 8%, 7%, 6%, 5%, 4%. 3%, 2%, or 1%) wt/v of the composition. In some embodiments, the one or more vitamins and minerals include: less than 9% wt/v of the composition. In some embodiments, the one or more vitamins and minerals include: less than 8% wt/v of the composition. In some embodiments, the one or more vitamins and minerals include: less than 7% wt/v of the composition. In some embodiments, the one or more vitamins and minerals include: less than 6% wt/v of the composition. In some embodiments, the one or more vitamins and minerals include: less than 5% N wt/v of the composition. In some embodiments, the one or more vitamins and minerals include: less than 4% wt/v of the composition. In some embodiments, the one or more vitamins and minerals include: less than 3% N wt/v of the composition. In some embodiments, the one or more vitamins and minerals include: less than 2% wt/v of the composition. In some embodiments, the one or more vitamins and minerals include: less than 1% wt/v of the composition.

In some embodiments, the one or more fatty acids and phospholipids (e.g., fish oil, tuna oil, squid oil, krill oil, algae oil, soy lecithin oil, and vegetable oil) include: from 0% to 10% (e.g., about 5%) wt/v of the composition. In some embodiments, the more fatty acids and phospholipids comprises about 5% w/v of the composition.

In some embodiments, the one or more fatty acids and phospholipids (e.g., fish oil, tuna oil, squid oil, krill oil, algae oil, soy lecithin oil, and vegetable oil) include: less than 10% (e.g., 9%, 8%, 7%, 6%, 5%, 4%, 3%, 2%, or 1%) wt/v of the composition. In some embodiments, the one or more fatty acids and phospholipids include: less than 9% wt/v of the composition. In some embodiments, the one or more fatty acids and phospholipids include: less than 8% wt/v of the composition. In some embodiments, the one or more fatty acids and phospholipids include: less than 7% wt/v of the composition. In some embodiments, the one or more fatty acids and phospholipids include: less than 6% wt/v of the composition. In some embodiments, the one or more fatty acids and phospholipids include: less than 5% wt/v of the composition. In some embodiments, the one or more fatty acids and phospholipids include: less than 4% wt/v of the composition. In some embodiments, the one or more fatty acids and phospholipids include: less than 3% wt/v of the composition. In some embodiments, the one or more fatty acids and phospholipids include: less than 2% wt/v of the composition. In some embodiments, the one or more fatty acids and phospholipids include: less than 1% wt/v of the composition.

In some embodiments, the one or more non-nutrient additives (e.g., a binder (e.g., carboxymethylcellulose), soy lecithin, and a mold inhibitor (e.g., calcium propionate)) include: from 0% to 10% (e.g., about 5%) wt/v of the composition. In some embodiments, the more non-nutrient additives comprises about 5% w/v of the composition.

In some embodiments, the one or more non-nutrient additives (e.g., a binder (e.g., carboxymethylcellulose), soy lecithin, and a mold inhibitor (e.g., calcium propionate)) include: less than 10% (e.g., 9%, 8%, 7%. 6%, 5%, 4%. 3%, 2%, or 1%) wt/v of the composition. In some embodiments, the one or more non-nutrient additives include: less than 9% wt/v of the composition. In some embodiments, the one or more non-nutrient additives include: less than 8% wt/v of the composition. In some embodiments, the one or more non-nutrient additives include: less than 7% wt/v of the composition. In some embodiments, the one or more non-nutrient additives include: less than 6% N wt/v of the composition. In some embodiments, the one or more non-nutrient additives include: less than 5% wt/v of the composition. In some embodiments, the one or more non-nutrient additives include: less than 4% wt/v of the composition. In some embodiments, the one or more non-nutrient additives include: less than 3% wt/v of the composition. In some embodiments, the one or more non-nutrient additives include: less than 2% wt/v of the composition. In some embodiments, the one or more non-nutrient additives include: less than 1% wt/v of the composition.

In some embodiments, the bacteriocin comprises from about 0.0001% to 20% (e.g., 0.001% to 19%, 0.01% to 18%, 0.1% to 17%, 1% to 16%, 5% to 15%, or about 10%) wt/v of the composition. For example, in some embodiments, the bacteriocin comprises from about 0.001% to 19% wt/v of the composition. In some embodiments, the bacteriocin comprises from about 0.01% to 18% wt/v of the composition. In some embodiments, the bacteriocin comprises from about 0.1% to 17% wt/v of the composition. In some embodiments, the bacteriocin comprises from about 1% to 16% wt/v of the composition. In some embodiments, the bacteriocin comprises from about 5% to 15% wt/v of the composition. In some embodiments, the bacteriocin comprises about 10% wt/v of the composition.

In some embodiments, the bacteriocin comprises about 0.01% wt/v of the composition.

In some embodiments, the bacteriocin comprises from about 1×10⁻³ mg/g to 200 mg/g of the composition.

In some embodiments, the Nisin comprises from about 0.0001% to 10% (e.g., 0.001% to 9%, 0.01% to 8%, 0.1% to 7%, 1% to 6%, or about 5%) wt/v of the composition. For example, in some embodiments, the Nisin comprises from about 0.001% to 9% wt/v of the composition. In some embodiments, the Nisin comprises from about 0.01% to 8% wt/v of the composition. In some embodiments, the Nisin comprises from about 0.1% to 7% wt/v of the composition. In some embodiments, the Nisin comprises from 1% to 6% wt/v of the composition. In some embodiments, the Nisin comprises from about 5% wt/v of the composition.

As used herein, “Nisin” can refer to pure Nisin or impure Nisin, wherein “impure” Nisin refers to a Nisin product containing about 10% pure Nisin (e.g., impure Nisin may include a Nisin product sold at 2.5% purity). In some embodiments, Nisin, as used herein, refers to pure Nisin. In some embodiments, Nisin, as used herein, refers to impure Nisin, wherein impure Nisin comprises about 10% pure Nisin.

In some embodiments, the Nisin is Nisin A, Nisin Z, or a combination thereof. Any suitable Nisin can be used.

In some embodiments, the Nisin comprises: less than 10% (e.g., 9%, 8%, 7%, 6%, 5%, 4%, 3%. 2%, or 1%) wt/v of the composition. In some embodiments, the Nisin comprises: less than 9% wt/v of the composition. In some embodiments, the Nisin comprises: less than 8% wt/v of the composition. In some embodiments, the Nisin comprises: less than 7% wt/v of the composition. In some embodiments, the Nisin comprises: less than 6% wt/v of the composition. In some embodiments, the Nisin comprises: less than 5% wt/v of the composition. In some embodiments, the Nisin comprises: less than 4% wt/v of the composition. In some embodiments, the Nisin comprises: less than 3% wt/v of the composition. In some embodiments, the Nisin comprises: less than 2% wt/v of the composition. In some embodiments, the Nisin comprises: less than 1% wt/v of the composition.

In some embodiments, a composition of the disclosure comprises: a bacteriocin (e.g., Nisin); and shrimp feed, wherein the shrimp feed comprises: anchovy meal, soybean meal, fish oil, tuna oil, wheat flour, krill meal, wheat gluten meal, choline chloride with choline, cholesterol, soy lecithin, Ca(H₂PO₄)₂, salt, Rovimix-stay-C 35, shrimp pre-mix, methionine, threonine, lysine, carboxymethylcellulose, and calcium propionate.

In some embodiments, a composition of the disclosure comprises: Nisin; and shrimp feed, wherein the shrimp feed comprises: anchovy meal, soybean meal, fish oil, tuna oil, wheat flour, krill meal, wheat gluten meal, choline chloride with choline, cholesterol, soy lecithin. Ca(H₂PO₄)₂, salt. Rovimix-stay-C 35, shrimp pre-mix, methionine, threonine, lysine, carboxymethylcellulose, and calcium propionate.

In some embodiments, the anchovy meal comprises about 26.5% (e.g., about 23%, about 24%, about 25%, about 26%, about 27%, about 28%, about 29%, or about 30%) wt/v of the shrimp feed; the soybean meal comprises about 30.4% (e.g., about 27%, about 28%, about 29%, about 30%, about 31%, about 32%, or about 33%) wt/v of the shrimp feed; the fish oil and tuna oil comprises about 1.8% (e.g., about 1%, about 2%, or about 3%) wt/v of the shrimp feed; the wheat flour comprises about 27.27% (about 23%, about 24%, about 25%, about 26%, about 27%, about 28%, about 29%, or about 30%) wt/v of the shrimp feed; the krill meal comprises about 2% (e.g., about 1%, about 2%, or about 3%) wt/v of the shrimp feed: the wheat gluten meal comprises about 3.5% (e.g., about 1%, about 2%, about 3%, about 4%, or about 5%) wt/v of the shrimp feed; the choline chloride with choline comprises about 1% (e.g., about 1%, about 2%, or about 3%) wt/v of the shrimp feed; the cholesterol comprises about 0.03% (e.g., about 1%) wt/v of the shrimp feed; the soy lecithin comprises about 1.5% (e.g., about 1%, about 2%, or about 3%) wt/v of the shrimp feed: the Ca(H₂PO₄)₂ comprises about 2% (e.g., about 1%, about 2%, or about 3%) wt/v of the shrimp feed: the salt comprises about 0.6% (e.g., about 1%) wt/v of the shrimp feed; the Rovimix-stay-C 35 comprises about 0.1% (e.g., about 1%) wt/v of the shrimp feed; the shrimp pre-mix comprises about 0.4% wt/v of the shrimp feed; the methionine comprises about 0.2% (e.g., about 1%) wt/v of the shrimp feed: the threonine comprises about 0.3% (e.g., about 1%) wt/v of the shrimp feed: the lysine comprises about 0.3% (e.g., about 1%) wt/v of the shrimp feed: the carboxymethylcellulose comprises about 2% (e.g., about 1%, about 2%, or about 3%) wt/v of the shrimp feed; and the calcium propionate comprises about 0.1% (e.g., about 1%) wt/v of the shrimp feed.

For example, in some embodiments, the anchovy meal comprises about 23% wt/v of the shrimp feed. In some embodiments, the anchovy meal comprises about 24% wt/v of the shrimp feed. In some embodiments, the anchovy meal comprises about 25% wt/v of the shrimp feed. In some embodiments, the anchovy meal comprises about 26% wt/v of the shrimp feed. In some embodiments, the anchovy meal comprises about 27% wt/v of the shrimp feed. In some embodiments, the anchovy meal comprises about 28% wt/v of the shrimp feed. In some embodiments, the anchovy meal comprises about 29% wt/v of the shrimp feed. In some embodiments, the anchovy meal comprises about 30% wt/v of the shrimp feed.

In some embodiments, the soybean meal comprises about 27% wt/v of the shrimp feed. In some embodiments, the soybean meal comprises about 28% wt/v of the shrimp feed. In some embodiments, the soybean meal comprises about 29% wt/v of the shrimp feed. In some embodiments, the soybean meal comprises about 30% wt/v of the shrimp feed. In some embodiments, the soybean meal comprises about 31% wt/v of the shrimp feed. In some embodiments, the soybean meal comprises about 32% wt/v of the shrimp feed. In some embodiments, the soybean meal comprises about 33% wt/v of the shrimp feed.

In some embodiments, the fish oil and tuna oil comprises about 1% wt/v of the shrimp feed. In some embodiments, the fish oil and tuna oil comprises about 2% wt/v of the shrimp feed. In some embodiments, the fish oil and tuna oil comprises about 3% wt/v of the shrimp feed.

In some embodiments, the wheat flour comprises about 23% wt/v of the shrimp feed. In some embodiments, the wheat flour comprises about 24% wt/v of the shrimp feed. In some embodiments, the wheat flour comprises about 25% wt/v of the shrimp feed. In some embodiments, the wheat flour comprises about 26% wt/v of the shrimp feed. In some embodiments, the wheat flour comprises about 27% wt/v of the shrimp feed. In some embodiments, the wheat flour comprises about 28% wt/v of the shrimp feed. In some embodiments, the wheat flour comprises about 29% wt/v of the shrimp feed. In some embodiments, the wheat flour comprises about 30% wt/v of the shrimp feed.

In some embodiments, the krill meal comprises about 1% wt/v of the shrimp feed. In some embodiments, the krill meal comprises about 2% wt/v of the shrimp feed. In some embodiments, the krill meal comprises about 3% wt/v of the shrimp feed.

In some embodiments, the gluten meal comprises about 1% wt/v of the shrimp feed. In some embodiments, the gluten meal comprises about 2% wt/v of the shrimp feed. In some embodiments, the gluten meal comprises about 3% wt/v of the shrimp feed. In some embodiments, the gluten meal comprises about 4% wt/v of the shrimp feed. In some embodiments, the gluten meal comprises about 5% wt/v of the shrimp feed.

In some embodiments, the choline chloride with choline comprises about 1% wt/v of the shrimp feed. In some embodiments, the choline chloride with choline comprises about 2% wt/v of the shrimp feed. In some embodiments, the choline chloride with choline comprises about 3% wt/v of the shrimp feed.

In some embodiments, the cholesterol comprises about 1% wt/v of the shrimp feed.

In some embodiments, the soy lecithin comprises about 1% wt/v of the shrimp feed. In some embodiments, the soy lecithin comprises about 2% wt/v of the shrimp feed. In some embodiments, the soy lecithin comprises about 3% wt/v of the shrimp feed.

In some embodiments, the Ca(H₂PO₄)₂ comprises about 1% wt/v of the shrimp feed. In some embodiments, the Ca(H₂PO₄)₂ comprises about 2% wt/v of the shrimp feed. In some embodiments, the Ca(H₂PO₄)₂ comprises about 3% wt/v of the shrimp feed.

In some embodiments, the salt comprises about 1% A wt/v of the shrimp feed.

In some embodiments, the Rovimix-stay-C 35 comprises about 1% wt/v of the shrimp feed.

In some embodiments, the shrimp pre-mix comprises about 1% wt/v of the shrimp feed.

In some embodiments, the methionine comprises about 1% wt/v of the shrimp feed.

In some embodiments, the threonine comprises about 1% wt/v of the shrimp feed.

In some embodiments, the lysine comprises about 1% wt/v of the shrimp feed.

In some embodiments, the carboxymethylcellulose comprises about 1% wt/v of the shrimp feed.

In some embodiments, the calcium propionate comprises about 1% wt/v of the shrimp feed.

Compositions described herein, in some embodiments, include combining the bacteriocin (e.g., Nisin) and shrimp feed. In some embodiments, combining comprises top-coating, extrusion, or mixing-in. For example, in some embodiments, combining comprises top-coating. In some embodiments, combining comprises extrusion. In some embodiments, combining comprises mixing-in.

In some embodiments, the bacteriocin (e.g., Nisin) comprises from 1×10⁻³ mg/g of the composition to 200 mg/g (e.g., from 1×10⁻³ to 100 mg/g, 1 mg/g to 90 mg/g, 2 mg/g to 80 mg/g, 3 mg/g to 70 mg/g. 4 mg/g to 60 mg/g, 5 mg/g to 50 mg/g, 10 mg/g to 40 mg/g, or 20 mg/g to 30 mg/g) of the composition. For example, in some embodiments, the bacteriocin (e.g., Nisin) comprises from 1×10⁻³ to 100 mg/g of the composition. In some embodiments, the bacteriocin (e.g., Nisin) comprises from 1 mg/g to 90 mg/g of the composition. In some embodiments, the bacteriocin (e.g., Nisin) comprises from 2 mg/g to 80 mg/g of the composition. In some embodiments, the bacteriocin (e.g., Nisin) comprises from 3 mg/g to 70 mg/g of the composition. In some embodiments, the bacteriocin (e.g., Nisin) comprises from 4 mg/g to 60 mg/g of the composition. In some embodiments, the bacteriocin (e.g., Nisin) comprises from 5 mg/g to 50 mg/g of the composition. In some embodiments, the bacteriocin (e.g., Nisin) comprises from 10 mg/g to 40 mg/g of the composition. In some embodiments, the bacteriocin (e.g., Nisin) comprises from 20 mg/g to 30 mg/g of the composition.

In some embodiments, the shrimp feed comprises about 99.99% wt/v of the composition; and the bacteriocin comprises about 0.010% w/v of the composition.

In some embodiments, the composition further comprises ethylenediaminetetraacetic acid (EDTA).

In some embodiments, the EDTA comprises from about 0.00010% to 2% (e.g., about 0.001 to 1% and about 0.01 to 0.05%) wt/v of the composition. In some embodiments, the EDTA comprises from about 0.001 to 1% w/v of the composition. In some embodiments, the EDTA comprises from about 0.01 to 0.05% w/v of the composition.

In some embodiments, the EDTA comprises about 0.01% wt/v of the composition.

In some embodiments, the EDTA comprises from about 0.001 mg/g to 20 mg/g of the composition.

In some embodiments, the: shrimp feed comprises about 99.98% wt/v of the composition: the bacteriocin comprises about 0.01% w/v of the composition; and the EDTA comprises about 0.01% wt/v of the composition.

It is to be understood that the above lists are not all-inclusive, and that an ingredient may fall within various categories (e.g., corn meal and wheat meal include protein).

In some embodiments, a composition of the disclosure does not include an antibiotic.

In some embodiments, the compositions of the disclosure are administered to a shrimp in need thereof (e.g., a farmed shrimp).

### Exemplary Compounds

Exemplary compositions of the disclosure comprise: a bacteriocin, wherein the bacteriocin comprises Nisin Z, and wherein the bacteriocin comprises about 0.01% w/v of the composition; and shrimp feed, wherein the shrimp feed comprises about 99.99% wt/v of the composition, and wherein: anchovy meal comprises about 26.5% wt/v of the shrimp feed; soybean meal comprises about 30.4% wt/v of the shrimp feed: fish oil and tuna oil comprise about 1.8% wt/v of the shrimp feed; wheat flour comprises about 27.27% wt/v of the shrimp feed; krill meal comprises about 2% wt/v of the shrimp feed; wheat gluten meal comprises about 3.5% wt/v of the shrimp feed; choline chloride with choline comprises about 1% wt/v of the shrimp feed; cholesterol comprises about 0.03% wt/v of the shrimp feed; soy lecithin comprises about 1.5% wt/v of the shrimp feed; Ca(H₂PO₄)₂ comprises about 2% wt/v of the shrimp feed; salt comprises about 0.6% wt/v of the shrimp feed; Rovimix-stay-C 35 comprises about 0.10% wt/v of the shrimp feed; shrimp pre-mix comprises about 0.4% wt/v of the shrimp feed; methionine comprises about 0.2% wt/v of the shrimp feed; threonine comprises about 0.3% wt/v of the shrimp feed; lysine comprises about 0.3% wt/v of the shrimp feed; carboxymethylcellulose comprises about 2% wt/v of the shrimp feed; and calcium propionate comprises about 0.10% w-t/v of the shrimp feed, wherein the composition prevents or treats a disease or one or more symptoms thereof caused by pathogenic Vibrio in a shrimp in need thereof.

The present disclosure also provides compositions comprising: a bacteriocin, wherein the bacteriocin comprises Nisin Z, and wherein the bacteriocin comprises about 0.010% w/v of the composition; EDTA, wherein the EDTA comprises about 0.010% wt/v of the composition; and shrimp feed, wherein the shrimp feed comprises about 99.98% wt/v of the composition, and wherein: anchovy meal comprises about 26.5% wt/v of the shrimp feed; soybean meal comprises about 30.4% wt/v of the shrimp feed: fish oil and tuna oil comprise about 1.8% wt/v of the shrimp feed; wheat flour comprises about 27.27% wt/v of the shrimp feed; krill meal comprises about 2% wt/v of the shrimp feed; wheat gluten meal comprises about 3.5% wt/v of the shrimp feed; choline chloride with choline comprises about 1% wt/v of the shrimp feed; cholesterol comprises about 0.03% wt/v of the shrimp feed; soy lecithin comprises about 1.5% wt/v of the shrimp feed; Ca(H₂PO₄)₂ comprises about 2% wt/v of the shrimp feed; salt comprises about 0.6% wt/v of the shrimp feed; Rovimix-stay-C 35 comprises about 0.1% wt/v of the shrimp feed; shrimp pre-mix comprises about 0.4% wt/v of the shrimp feed; methionine comprises about 0.2% wt/v of the shrimp feed; threonine comprises about 0.3% wt/v of the shrimp feed; lysine comprises about 0.3% wt/v of the shrimp feed; carboxymethylcellulose comprises about 2% wt/v of the shrimp feed; and calcium propionate comprises about 0.10% wt/v of the shrimp feed, wherein the composition prevents or treats a disease or one or more symptoms thereof caused by pathogenic Vibrio in a shrimp in need thereof.

### Methods of Treatment

Using the compositions described herein are methods of treating a disease or one or more symptoms thereof caused by pathogenic Vibrio (e.g., Vibrio parahaemolyticus, Vibrio alginolyticus, Vibrio vulnificus, Vibrio cholera, Vibrio harveyi, Vibrio fischeri, Vibrio anguillarum, Vibrio spledidus, Vibrio nerves, Vibrio tubiashii, Vibrio campbellii, Vibrio aestuarianus, Vibrio pelagius, Vibrio mediterranei, Vibrio diazotrophicus, Vibrio damselae, Vibrio mimicus, and Vibrio cholera) in a shrimp in need thereof (e.g., a farmed shrimp), wherein the method comprises administering a composition of the disclosure.

For example, in some embodiments, the disease comprises: acute hepatopancreatic necrosis disease (AHPND), vibriosis, loose shell syndrome (LSS), tail necrosis, shell disease, red disease, white gut disease (WGD), white tail disease, white feces disease (WFD), luminous bacterial disease, and necrotizing hepatopancreatitis. In some embodiments, the disease is AHPND. In some embodiments, the disease is vibriosis. In some embodiments, the disease is LSS. In some embodiments, the disease is tail necrosis. In some embodiments, the disease is shell disease. In some embodiments, the disease is red disease. In some embodiments, the disease is WGD. In some embodiments, the disease is white tail disease. In some embodiments, the disease is WFD. In some embodiments, the disease is luminous bacterial disease. In some embodiments, the disease is necrotizing hepatopancreatitis.

Also provided herein are methods of reducing a concentration of pathogenic Vibrio (e.g., Vibrio parahaemolyticus, Vibrio alginolyticus, Vibrio vulnificus, Vibrio cholera, Vibrio harveyi, Vibrio fischeri, Vibrio anguillarum, Vibrio spledidus, Vibrio nerves, Vibrio tubiashii, Vibrio campbellii, Vibrio aestuarianus, Vibrio pelagius, Vibrio mediterranei, Vibrio diazotrophicus, Vibrio damselae, Vibrio mimicus, and Vibrio cholera) in a shrimp in need thereof (e.g., a farmed shrimp), wherein the method comprises administering a composition of the disclosure.

For example, in some embodiments, the pathogenic Vibrio is Vibrio parahaemolyticus. In some embodiments, the pathogenic Vibrio is Vibrio alginolyticus. In some embodiments, the pathogenic Vibrio is Vibrio vulnificus. In some embodiments, the pathogenic Vibrio is Vibrio cholera. In some embodiments, the pathogenic Vibrio is Vibrio harveyi. In some embodiments, the pathogenic Vibrio is Vibrio fischeri. In some embodiments, the pathogenic Vibrio is Vibrio anguillarum. In some embodiments, the pathogenic Vibrio is Vibrio spledidus. In some embodiments, the pathogenic Vibrio is Vibrio nerves. In some embodiments, the pathogenic Vibrio is Vibrio tubiashii. In some embodiments, the pathogenic Vibrio is Vibrio campbellii. In some embodiments, the pathogenic Vibrio is Vibrio aestuarianus. In some embodiments, the pathogenic Vibrio is Vibrio pelagius. In some embodiments, the pathogenic Vibrio is Vibrio mediterranei. In some embodiments, the pathogenic Vibrio is Vibrio diazotrophicus. In some embodiments, the pathogenic Vibrio is Vibrio damselae. In some embodiments, the pathogenic Vibrio is Vibrio mimicus. In some embodiments, the pathogenic Vibrio is Vibrio cholera.

In some embodiments, the compositions of the disclosure are administered to a shrimp in need thereof.

In accordance with the methods described herein, the described compositions herein may be formulated for administration, for example, by oral administration, and the pharmaceutical compositions formulated accordingly.

According to a particular embodiment, the pharmaceutically acceptable carrier of a composition useful in the practice of the invention is formulated for oral administration.

The pharmaceutical compositions containing compound described herein may be manufactured in a manner that is generally known, e.g., by means of top-coating, extrusion, conventional mixing, dissolving, granulating, levigating, emulsifying, encapsulating, entrapping, or lyophilizing processes. Pharmaceutical compositions may be formulated in a conventional manner using one or more pharmaceutically acceptable carriers including excipients and/or auxiliaries that facilitate processing of the compounds into preparations that can be used pharmaceutically. It is appreciated that the appropriate formulation is dependent upon the route of administration chosen.

Oral compositions generally include an inert diluent or an edible pharmaceutically acceptable carrier. They can be enclosed in gelatin capsules or compressed into tablets. For the purpose of oral therapeutic administration, the compounds described herein can be incorporated with excipients and used in the form of tablets, troches, or capsules. Oral compositions can also be prepared using a fluid carrier for use as a mouthwash, Wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, an active ingredient; a binder such as microcrystalline cellulose, gum tragacanth or gelatin; an excipient such as starch or lactose, a disintegrating agent such as alginic acid. Primogel, or corn starch: a lubricant such as magnesium stearate: a glidant such as colloidal silicon dioxide; a sweetening agent such as sucrose or saccharin; or a flavoring agent such as peppermint, methyl salicylate, or orange flavoring.

It is advantageous to formulate oral compositions in dosage unit form for ease of administration and uniformity of dosage. Dosage unit form as used herein refers to physically discrete units suited as unitary dosages for the subject to be treated; each unit containing a predetermined quantity of the active ingredient calculated to produce the desired therapeutic effect in association with the required pharmaceutical carrier. The specification for the dosage unit forms are dictated by and directly dependent on the unique characteristics of the active ingredient and the particular therapeutic effect to be achieved.

In therapeutic applications, the dosages of the pharmaceutical compositions used in accordance with the disclosure vary depending on the agent, the age, weight, and clinical condition of the recipient subject, and the experience and judgment of the clinician or practitioner administering the therapy, among other factors affecting the selected dosage. Generally, the dose should be sufficient to result in slowing, and preferably regressing, the symptoms of the disease or disorder disclosed herein and preferably causing complete regression of the disease or disorder. An effective amount of a pharmaceutical agent is that which provides an objectively identifiable improvement as noted by the clinician or other qualified observer.

It is to be understood that the pharmaceutical compositions can be included in feed, in a container, pack, or dispenser together with instructions for administration.

In some embodiments, the shrimp in need thereof is or has not been administered an antibiotic. For example, in some embodiments, the shrimp in need thereof is not being administered an antibiotic. In some embodiments, the shrimp in need thereof has not been administered an antibiotic.

In some embodiments, upon administration of composition of the disclosure, a concentration of pathogenic Vibrio is reduced in the shrimp in need thereof.

In some embodiments, upon administration of composition of the disclosure, one or more symptoms (e.g., lethargy, anorexia, slow growth, an empty digestive tract, pale-to-white hepatopancreases, reddish body discoloration, yellowing gill tissue, appearance of white patches in abdominal muscle, melanisation, granulomatous encapsulation, luminescence, a soft shell, melanised spots or streaks visible within the hepatopancreas, and atrophy of the hepatopancreas) caused by pathogenic Vibrio (e.g., Vibrio parahaemolyticus, Vibrio alginolyticus, Vibrio vulnificus, Vibrio cholera, Vibrio harveyi, Vibrio fischeri, Vibrio anguillarum, Vibrio spledidus, Vibrio nerves, Vibrio tubiashii, Vibrio campbellii, Vibrio aestuarianus, Vibrio pelagius, Vibrio mediterranei, Vibrio diazotrophicus, Vibrio damselae, Vibrio mimicus, and Vibrio cholera) is reduced in the shrimp in need thereof. For example, in some embodiments, upon administration of composition of the disclosure, lethargy caused by pathogenic Vibrio is reduced in the shrimp in need thereof. In some embodiments, upon administration of composition of the disclosure, anorexia caused by pathogenic Vibrio is reduced in the shrimp in need thereof. In some embodiments, upon administration of composition of the disclosure, slow growth caused by pathogenic Vibrio is reduced in the shrimp in need thereof. In some embodiments, upon administration of composition of the disclosure, an empty digestive tract caused by pathogenic Vibrio is reduced in the shrimp in need thereof. In some embodiments, upon administration of composition of the disclosure, pale-to-white hepatopancreases caused by pathogenic Vibrio is reduced in the shrimp in need thereof. In some embodiments, upon administration of composition of the disclosure, reddish body discoloration caused by pathogenic Vibrio is reduced in the shrimp in need thereof. In some embodiments, upon administration of composition of the disclosure, yellowing gill tissue caused by pathogenic Vibrio is reduced in the shrimp in need thereof. In some embodiments, upon administration of composition of the disclosure, appearance of white patches in abdominal muscle caused by pathogenic Vibrio is reduced in the shrimp in need thereof. In some embodiments, upon administration of composition of the disclosure, melanisation caused by pathogenic Vibrio is reduced in the shrimp in need thereof. In some embodiments, upon administration of composition of the disclosure, granulomatous encapsulation caused by pathogenic Vibrio is reduced in the shrimp in need thereof. In some embodiments, upon administration of composition of the disclosure, luminescence caused by pathogenic Vibrio is reduced in the shrimp in need thereof. In some embodiments, upon administration of composition of the disclosure, a soft shell caused by pathogenic Vibrio is reduced in the shrimp in need thereof. In some embodiments, upon administration of composition of the disclosure, melanised spots or streaks visible within the hepatopancreas caused by pathogenic Vibrio is reduced in the shrimp in need thereof. In some embodiments, upon administration of composition of the disclosure, atrophy of the hepatopancreas caused by pathogenic Vibrio is reduced in the shrimp in need thereof.

In some embodiments, upon administration of composition of the disclosure, a microbiome of the shrimp in need thereof is modified relative to a suitable control.

In some embodiments, the shrimp in need thereof is a farmed shrimp.

### EXAMPLES

Below are examples of specific embodiments for carrying out the present invention. The examples are offered for illustrative purposes only and are not intended to limit the scope of the present invention in any way.

### Example 1. Characterizing the Minimum Inhibitory Concentration of Nisin

This Example describes the materials and methods, and results of a serial dilution experiment to determine the Minimum Inhibitory Concentration (MIC) of Nisin in reducing a concentration of pathogenic Vibrio.

### Materials and Methods

On the day before the experiment, Vibrio stock were prepared by sterilizing an inoculation loop, cooling the product, and extracting cells. Such extract was incubated at 37° C. for 16 to 20 hours. The density of cells was normalized to a cell density of approximately 10⁷ to 10⁸ CFU/mL and 100 uL lawns were prepared at a cell density of about 10⁶ to 10⁷ CFU using sterile glass beads. Four lawns were made so that each Nisin dilution could be tested on two identical plates.

5 μL of two-fold dilutions (See Table 1, below) of Nisin (Immucell; 99% pure), or shrimp feed (without Nisin), citric acid, or DH₂0 negative controls, respectively, were spotted on the plates, which were incubated at 37° C. for 16 to 20 hours. The minimum inhibitory concentration was determined. In particular, plates were characterized based upon the presence (0) or absence (1) of Vibrio growth.

TABLE 1
Serial Dilutions of Nisin alone or Nisin with Shrimp Feed

| Dilution Level | Nisin Concentration (mg/mL) |
|---|---|
| Original | 4.23 |
| 1 | 2.12 |
| 2 | 1.06 |
| 3 | 0.529 |
| 4 | 0.264 |
| 5 | 0.132 |
| 6 | 0.0661 |
| 7 | 0.0330 |
| 8 | 0.0165 |
| 9 | 0.00826 |
| 10 | 0.00413 |
| 11 | 0.00207 |
| 12 | 0.00103 |
| 13 | 0.000515 |
| 14 | 0.0002575 |
| 15 | 0.0001287 |

### Results

As presented in Tables 2 and 3, below, the MIC of Nisin was calculated by Vibrio species. In all species of Vibrio, Nisin had a significantly smaller MIC than the negative control. As evidenced by this data, Nisin is highly effective at reducing the concentration of many pathogenic Vibrio species.

TABLE 2
The MIC of Nisin in ug/mL

| | Bacteriocin | Negative Control |
|---|---|---|
| Species | MIC (ug/mL) | MIC (ug/mL) |
| Vibrio parahaemolyticus^{a} | 8.3 | >1.00E+06 |
| Vibrio parahaemolyticus^{a} | 8.3 | >1.00E+06 |
| Vibrio parahaemolyticus^{a} | 528.8 | >1.00E+06 |
| Vibrio alginolyticus^{b} | 2,115.0 | >1.00E+06 |
| Vibrio parahaemolyticus^{a} | 1,057.5 | >1.00E+06 |
| Vibrio parahaemolyticus^{a} | 1,057.5 | >1.00E+06 |
| Vibrio cholerae^{c} | 1,057.5 | >1.00E+06 |
| Vibrio alginolyticus^{b} | 8.3 | >1.00E+06 |
| Vibrio parahaemolyticus^{a} | 33.1 | >1.00E+06 |
| Vibrio alginolyticus^{b} | 66.1 | >1.00E+06 |
| Vibrio cholerae^{c} | 8.3 | >1.00E+06 |
| Vibrio parahaemolyticus^{a} | 1,057.5 | >1.00E+06 |
| Vibrio parahaemolyticus^{a} | 66.1 | >1.00E+06 |
| Vibrio parahaemolyticus^{a} | 528.8 | >1.00E+06 |
| Vibrio cholerae^{c} | 2,115.0 | >1.00E+06 |
| Vibrio cholerae^{c} | 132.2 | >1.00E+06 |
| Vibrio parahaemolyticus^{a} | 528.8 | >1.00E+06 |
| Mean | 610.4 | >1.00E+06 |
| Vibrio parahaemolyticus (^{a}) Mean | 487.4 | >1.00E+06 |
| Vibrio alginolyticus (^{b}) Mean | 729.8 | >1.00E+06 |
| Vibrio cholerae (^{c}) Mean | 828.2 | >1.00E+06 |

TABLE 3
The MIC of Nisin in % weight per volume

| | Bacteriocin | Negative Control |
|---|---|---|
| Species | MIC (wt/v %) | MIC (wt/v %) |
| Vibrio parahaemolyticus^{a} | 0.01 | >100.00 |
| Vibrio parahaemolyticus^{a} | 0.01 | >100.00 |
| Vibrio parahaemolyticus^{a} | 0.53 | >100.00 |
| Vibrio alginolyticus^{b} | 2.12 | >100.00 |
| Vibrio parahaemolyticus^{a} | 1.06 | >100.00 |
| Vibrio parahaemolyticus^{a} | 1.06 | >100.00 |
| Vibrio cholerae^{c} | 1.06 | >100.00 |
| Vibrio alginolyticus^{b} | 0.01 | >100.00 |
| Vibrio parahaemolyticus^{a} | 0.03 | >100.00 |
| Vibrio alginolyticus^{b} | 0.07 | >100.00 |
| Vibrio cholerae^{c} | 0.01 | >100.00 |
| Vibrio parahaemolyticus^{a} | 1.06 | >100.00 |
| Vibrio parahaemolyticus^{a} | 0.07 | >100.00 |
| Vibrio parahaemolyticus^{a} | 0.53 | >100.00 |
| Vibrio cholerae^{c} | 2.12 | >100.00 |
| Vibrio cholerae^{c} | 0.13 | >100.00 |
| Vibrio parahaemolyticus^{a} | 0.53 | >100.00 |
| Mean | 0.61 | >100.00 |
| Vibrio parahaemolyticus (^{a}) Mean | 0.49 | >100.00 |
| Vibrio alginolyticus (^{b}) Mean | 0.73 | >100.00 |
| Vibrio cholerae (^{c}) Mean | 0.83 | >100.00 |

### Example 2. Shrimp Feed with Nisin Reduces Pathogenesis and Promotes Survival of Shrimp

This Example describes the materials, methods, and results of a feed-and-survival assay in shrimp treated with a composition of the disclosure, including the bacteriocin Nisin and shrimp feed.

### Methods

### Shrimp

Specific Pathogen Free (SPF) P. vannamei at an average weight of 1 g to 2 g, were obtained from a commercial shrimp producer and reared. A total of 300 shrimp were transferred into 15 90 L glass aquaria, each aquarium stocked with 20 shrimp.

### Diet Preparation

Shrimp feed diets was prepared at X, 10X, and 100X (0.84 mg/g, 8.4 mg/g, and 84 mg/g Nisin, respectively) estimated concentrations of the bacteriocin Nisin. The bacteriocin additive was combined with a 40% protein commercial shrimp premix (Rangen Inc., Buhl, Idaho), carboxymethyl cellulose at a 3% inclusion rate, and up to 40% w/v water. Exemplary components of a typical 35% protein shrimp diet were wheat flour (35%), soybean meal (20%) and fishmeal (25%). The mixture was then cold-extruded through a meat grinder to produce a 3 mm diameter size pellet strand. The resulting feed strands were dried overnight at 40° C. and broken into appropriately-sized pellets. A total of 100 g of each diet was use for this study. In all three experimental groups a minimum of 9.324 g of Nisin was used to produce the respective feed conditions.

The negative and positive control tanks were fed Rangen's commercially pelleted shrimp diet without additive.

### Experimental Design

The commercially prepared shrimp diet from juvenile Penaeus (Litopenaeus) vannameia was prepared with a bacteriocin additive at three concentrations (0.84 mg/g, 8.4 mg/g, and 84 mg/g Nisin). Experimental shrimp were orally exposed for 7 to Vibrio parahaemolyticus, an agent which causes Acute Hepatopancreatic Necrosis Disease (AHPND) to determine if the feed additives (provided during and after exposure) would affect survival when compared to the group that was fed an untreated diet.

All tanks were covered to reduce the risk of mortality due to shrimp jumping out. Each group of tanks was outfitted with its own equipment (e.g., nets and buckets). Each tank contained sufficient aeration and was outfitted with a pre-conditioned biological filter. Water quality tests to characterize temperature, nitrite and total ammonia were performed on tanks weekly.

Nine tanks (three per Nisin concentration) were used to house shrimp that were fed the bacteriocin-containing diet, while three tanks were used to house shrimp fed the control diet without additive. In both cases, the shrimp were fed their respective diet, followed by the AHPND challenge. The challenge was provided on 7 days at 5% of body weight per tank. Three additional tanks were used as a negative control for environmental conditions and were used to house shrimp fed the control diet and not challenged with AHPND. Additionally, the negative control tanks were be fed before the AHPND challenged tanks to reduce the risk of cross contamination. See Table 4 for study design.

TABLE 4
Experimental Groups of AHPND Challenge

| | | | AHPND |
|---|---|---|---|
| | Diet/ | Tank | Challenge |
| Tank Number | Concentration | Definition | (y/n) |
| Tank 1 | Control | Negative Control | No |
| Tank 2 | Control | Negative Control | No |
| Tank 3 | Control | Negative Control | No |
| Tank 4 | Control | AHPND Positive | Yes |
| | | Control | |
| Tank 5 | Control | AHPND Positive | Yes |
| | | Control | |
| Tank 6 | Control | AHPND Positive | Yes |
| | | Control | |
| Tank 7 | Feed A at 0.84 mg/g | AHPND Challenge | Yes |
| Tank 8 | Feed A at 0.84 mg/g | AHPND Challenge | Yes |
| Tank 9 | Feed A at 0.84 mg/g | AHPND Challenge | Yes |
| Tank 10 | Feed A at 8.4 mg/g | AHPND Challenge | Yes |
| Tank 11 | Feed A at 8.4 mg/g | AHPND Challenge | Yes |
| Tank 12 | Feed A at 8.4 mg/g | AHPND Challenge | Yes |
| Tank 13 | Feed A at 84 mg/g | AHPND Challenge | Yes |
| Tank 14 | Feed A at 84 mg/g | AHPND Challenge | Yes |
| Tank 15 | Feed A at 84 mg/g | AHPND Challenge | Yes |

### AHPND Challenge

Each tank scheduled for the AHPND challenge was subjected to an immersion challenge. Tryptic Soy Broth +2% (TSB+) sodium chloride was inoculated with a consistently virulent strain of Vibrio parahaemolyticus (strain 13-028A/3), incubated for 18 hours, and added directly into each challenge tank. The bacterial suspension was added into each tank to achieve the bacterial density measured by optical density absorbance (OD₆₀₀ nm) at approximately 9.0 cells/mL.

### AHPND Sampling and Observation:

Two animals were preserved in Davidson's AFA fixative prior to the start of the study to document the SPF health status of the population by histology.

All tanks were checked twice a day for dead or moribund animals. During the study, any mortalities were removed from the tank and frozen at −20° C. Up to two moribund animals from each diet group, including the positive control tanks, were preserved in Davidson's AFA fixative and processed for routine histology to confirm an AHPND infection (up to 12 P. vannamei processed for histology during the challenge period).

The study was terminated after 14 days (7 days pre-feeding followed by a 7-day AHPND challenge). At termination, all live animals were counted as survivors with up to 2 survivors per diet group preserved for histology (for a total of 22 samples for histology).

Any survivors not accounted for at the termination of the study were assumed to be cannibalized.

### Results

The study revealed a statistically significant difference in cumulative survival between the positive control group (no treatment, challenged) and group receiving the Nisin 0.84 mg/g dose (see Table 5, FIG. 1, and FIGS. 2A-2E). The 0.84 mg/g group had 100% survival of AHPND-challenged shrimp (there were 4 deaths pre-challenge i.e., a 93% survival in shrimp overall) compared to a 35% survival in the positive controls. This difference represents a statistically significant 265% increase in relative survival of AHPND-challenged shrimp. The 8.4 mg/g group had a 68% survival rate, which was significantly higher than the positive controls. There was not a single death in the 84 mg/g treatment group (100% survival), which was also statistically significant as compared to the positive controls.

Taken together, these results demonstrate that compositions herein, including Nisin and shrimp feed (e.g., about 35% wheat flour, about 20% soybean meal, and about 25% fishmeal) at a dose of as low as 0.84 mg/g inclusion of Nisin produces a significant increase in shrimp survival, clearly identifying that the compositions herein can be used to prevent or treat a disease or one or more symptoms thereof caused by pathogenic Vibrio in a shrimp in need thereof.

TABLE 5
Cumulative Survival of AHPND Challenge

| | Treat | Mortalities by Day | | | | | | | | | | | | | | | | | | % | Group |
|---|---|---|---|---|---|---|---|---|---|---|---|---|---|---|---|---|---|---|---|---|---|
| Tank | ment | 0 | 1 | 2 | 3 | 4 | 5 | 6 | 7 | 8 | 9 | 10 | 11 | 12 | 13 | 14 | 15 | 16 | Survivors | Survival | Survival |
| 1 | Neg | 0 | 0 | 0 | 0 | 0 | 0 | 0 | 0 | 0 | 0 | 0 | 0 | 0 | 0 | 0 | 0 | 0 | 20 | 100% | 98% |
| | Cont | | | | | | | | | | | | | | | | | | | | |
| 2 | Neg | 0 | 0 | 0 | 0 | 0 | 0 | 0 | 0 | 0 | 0 | 0 | 0 | 0 | 0 | 0 | 0 | 0 | 20 | 100% | |
| | Cont | | | | | | | | | | | | | | | | | | | | |
| 3 | Neg | 0 | 0 | 0 | 0 | 0 | 0 | 0 | 0 | 0 | 0 | 0 | 0 | 0 | 0 | 0 | 0 | 0 | 19 | 95% | |
| | Cont | | | | | | | | | | | | | | | | | | | | |
| 4 | Pos | 0 | 0 | 0 | 0 | 0 | 0 | 0 | 0 | 12 | 4 | 1 | 0 | 1 | 0 | 0 | 0 | 1 | 1 | 5% | 35% |
| | Cont | | | | | | | | | | | | | | | | | | | | |
| 5 | Pos | 0 | 0 | 0 | 0 | 0 | 0 | 0 | 0 | 0 | 0 | 0 | 0 | 0 | 0 | 0 | 0 | 0 | 20 | 100% | |
| | Cont | | | | | | | | | | | | | | | | | | | | |
| 6 | Pos | 0 | 0 | 0 | 0 | 0 | 0 | 0 | 0 | 14 | 4 | 2 | | | | | | | 0 | 0% | |
| | Cont | | | | | | | | | | | | | | | | | | | | |
| 7 | 1X | 0 | 0 | 0 | 0 | 0 | 0 | 0 | 0 | 0 | 0 | 0 | 0 | 0 | 0 | 0 | 0 | 0 | 19 | 95% | 93% |
| 8 | 1X | 0 | 0 | 0 | 0 | 0 | 0 | 0 | 0 | 0 | 0 | 0 | 0 | 0 | 0 | 0 | 0 | 0 | 19 | 95% | |
| 9 | 1X | 0 | 2 | 0 | 0 | 0 | 0 | 0 | 0 | 0 | 0 | 0 | 0 | 0 | 0 | 0 | 0 | 0 | 18 | 90% | |
| 10 | 10X | 0 | 0 | 0 | 0 | 0 | 0 | 0 | 0 | 0 | 1 | 0 | 0 | 0 | 0 | 0 | 0 | 0 | 17 | 85% | 68% |
| 11 | 10X | 0 | 0 | 0 | 0 | 0 | 0 | 0 | 0 | 0 | 0 | 0 | 0 | 0 | 0 | 0 | 0 | 0 | 15 | 75% | |
| 12 | 10X | 0 | 0 | 0 | 0 | 0 | 0 | 0 | 0 | 2 | 3 | 2 | 3 | 1 | 0 | 0 | 0 | 0 | 9 | 45% | |
| 13 | 100X | 0 | 0 | 0 | 0 | 0 | 0 | 0 | 0 | 0 | 0 | 0 | 0 | 0 | 0 | 0 | 0 | 0 | 20 | 100% | 100% |
| 14 | 100X | 0 | 0 | 0 | 0 | 0 | 0 | 0 | 0 | 0 | 0 | 0 | 0 | 0 | 0 | 0 | 0 | 0 | 20 | 100% | |
| 15 | 100X | 0 | 0 | 0 | 0 | 0 | 0 | 0 | 0 | 0 | 0 | 0 | 0 | 0 | 0 | 0 | 0 | 0 | 20 | 100% | |

### Example 3. Shrimp Feed with Nisin can be Used to Reduce Pathogenesis and Promotes Survival of Shrimp

This Example describes an exemplary composition of the disclosure that can be used to prevent or treat a disease or one or more symptoms thereof caused by pathogenic Vibrio in a shrimp in need thereof e.g., farmed shrimp.

A composition of the disclosure (e.g., a bacteriocin (e.g., Nisin) and shrimp feed) can be prepared according to the general outline described in Example 2 with, for example, the ingredients outlined in Table 6 and Table 7.

TABLE 6
Exemplary Components of a Diet Composition

| Ingredients | % (wt/v) |
|---|---|
| Fish Meal (e.g., anchovy) | 26.50 |
| Soy bean meal, 48% CP, solvent extracted | 30.40 |
| Fish oil, Tuna oil, crude, Co-products | 1.80 |
| Wheat, flour | 27.35 |
| Krill meal | 2.00 |
| Wheat gluten meal, 78% CP | 3.50 |
| Choline chloride, 60% choline | 1.00 |
| Cholesterol, feed grade | 0.03 |
| Soy lecithin | 1.50 |
| Mono calcium phosphate, MCP, Ca(H₂PO4)₂ | 2.00 |
| Salt, NaCl | 0.64 |
| Rovimix-stay-C 35, ascorbyl-monophosphate | 0.13 |
| Shrimp Premix (Mix of Vitamin + Minerals) | 0.38 |
| DL-Methionine | 0.16 |
| L-Threonine | 0.30 |
| L-Lysine | 0.25 |
| Carboxymethylcellulose (CMC) | 2.00 |
| Mold inhibitor (calcium propionate) | 0.07 |
| Total | 100.00 |

TABLE 7
Exemplary Components and Characteristics of a Diet Composition

| Component | Amount |
|---|---|
| Moisture (%) | 10 |
| Crude protein (%) | 40.43 |
| Crude lipid (%) | 6.83 |
| Crude fiber (%) | 1.25 |
| Lysine (%) | 2.69 |
| Methionine (%) | 0.98 |
| Threonine (%) | 1.80 |
| Cholesterol (mg/kg) | 1482.97 |
| Phospholipids (%) | 2.12 |

### Example 4. Various Nisins can be Used to Reduce Pathogenesis and Promote Survival of Shrimp

This Example describes the efficacy of various Nisins: Nisin Z, Nisin A, and ImmuCell Nisin A. This experiment assessed the safety and antimicrobial efficacy of various Nisin formulations in treating Vibrio parahaemolyticus infections, a common pathogen in aquaculture environments. Brine shrimp (Artemia salina) were utilized as a model organism to simulate potential impacts on Pacific whiteleg shrimp (Penaeus vannamei). Toxicity trials (data not shown) showed that a reduction in pH, that results when Nisin is in citric acid buffer solution, is responsible for mortality that is not related to the peptide Nisin itself.

In additional toxicity trials, infra, the best Nisin candidates were screened and selected.

### Materials and Methods

### Materials

ImmuCell Nisin A (96% pure) suspended in citric acid buffer (pH-3.4) at 4.86 mg/mL (ImmuCell, Portland, ME); Nisin A (Nissen®-A; 10% pure powder, Siveele, Breda, Netherlands): Nisin Z (26.7% pure) powder (Handry, Brussels, Belgium): Citric Acid Buffer (CAB) pH=3.4; Distilled H2O (dH2O); Instant Ocean (Instant Ocean Spectrum Brands, Blacksburg. VA): Artemia salina eggs: 60 mm Petri Dishes; Laboratory control strains of Staphylococcus aureus and Escherichia coli (“SA1” and “EC-18,” respectively): 15 mL Falcon Tubes; and Slide-A-Lyzer Dialysis Flask, 2K MWCO, 250 mL (Thermo Scientific, Rockford, IL) were used.

### Preparation of Nisin Solutions:

Nisin variants (Nisin A, Nisin Z, and ImmuCell Nisin A) were dissolved in either citric acid buffer (CAB, pH 3.4) for Nisin A or dH2O for Nisin Z to make a stock solution of 4.86 mg/mL. Serial dilutions were performed to achieve the different concentrations (4.86 mg/mL to 0.005 mg/mL) for the experiments.

### Brine Shrimp Cultivation and Exposure:

Artemia salina eggs were hatched in artificial seawater (Instant Ocean) under controlled conditions. Newly hatched brine shrimp were then exposed to varying concentrations of each Nisin solution in experimental tanks. A separate set of tanks was used for each type of Nisin formulation.

### Antimicrobial Efficacy Assessment:

Parallel to the toxicity tests, antibacterial efficacy was evaluated by applying Nisin solutions (before and after basic purification via Slide-A-Lyzer Dialysis) onto agar plates pre-inoculated with laboratory control strains Staphylococcus aureus (“SA1”) and Escherichia coli (“EC-18”). Zones of inhibition were measured 24 hours post-incubation at 37° C.

### Experimental Design

### Control Setup

Control tanks (treated with either CAB or saline water) were prepared to observe the natural behavior and survival rate of Artemia salina without Nisin exposure.

### Treatment Groups

Brine shrimp were divided into different tanks for exposure to varying concentrations (2.43 mg/mL, 0.608 mg/mL, 0.152 mg/mL, and 0.038 mg/mL) of Nisin A, Nisin Z, and ImmuCell Nisin A.

### Data Collection

Mortality and behavior of the brine shrimp were recorded at 24 and 48 hours post-exposure. For the antimicrobial tests, the diameter of inhibition zones on agar plates containing the laboratory control strains were measured and recorded.

### Results

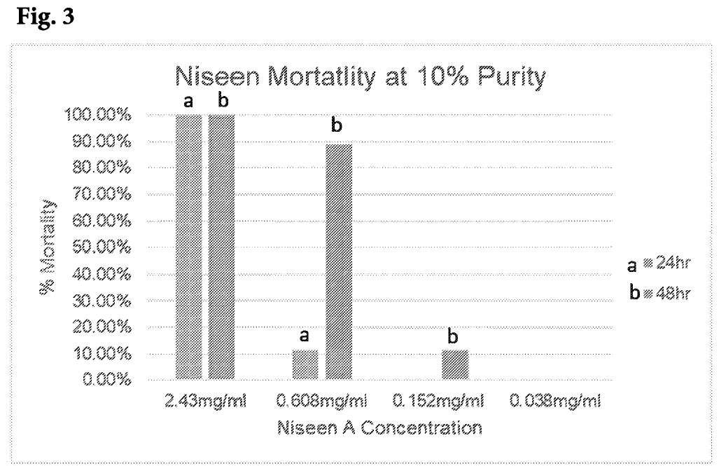

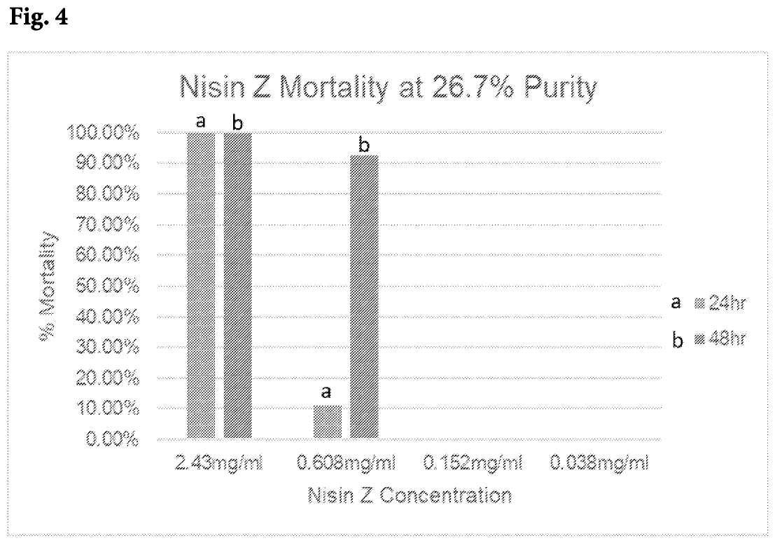

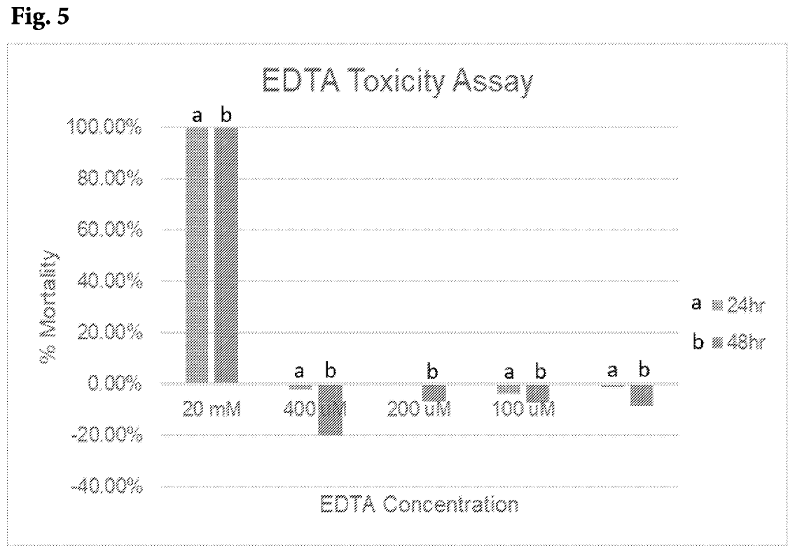

Immucell Nisin at all dilutions (4.86 mg/mL-0.005 mg/mL) tested in SAT and EC-18 had clear zones of inhibition, Nisin A (pre-purification) showed killing in SA1 and EC-18 down to a concentration 0.019 mg/mL, reduced growth at 0.009 mg/mL, and no killing at 0.005 mg/mL (FIG. 3), Nisin Z (pre-purification) showed killing in SA1 and EC-18 down to a concentration 0.019 mg/mL, reduced growth at 0.009 mg/mL, and no killing at 0.005 mg/mL (FIG. 4). These results demonstrated that Nisin Z and Nisin A have comparable efficacy in reducing mortality in shrimp, despite Nisin Z's purity of 26.7% compared to Nisin A's purity of 10% (there are fewer impurities that need to be taken out of Nisin Z). Additionally, Nisin Z went into suspension fully with dH₂O as a solvent (which is important to keep the pH near neutral for the brine shrimp) while Nisin A did not go fully into suspension.

In the unpurified state and suspended in dH2O, Nisin Z showed no toxicity at concentrations of 0.152 mg/mL to 0.038 mg/mL at 48 hours, whereas Nisin A showed mortality at 0.152 mg/mL. The drastic increase in mortality at 0.608 mg/mL, without being bound by theory, may have been the result of impurities surpassing a particular threshold where the brine shrimp cannot survive. The amount of powder used for Nisin A compared to Nisin Z may also have been a factor. For example, at 0.152 mg/mL drug concentration Niseen A required 1.52 mg of powder filled with impurities and Nisin Z at the same 0.152 mg/mL drug concentration required only 0.57 mg of powder filled with impurities. Based on this, Nisin Z was selected, though both were observed to be efficacious.

Next, the zones of inhibition for Nisin Z pre- and 48 hours post-2 kDa dialysis membrane purification were spotted onto a lawn of a control strain (SA1). In result, it was observed that same sized zones were produced with no loss of Nisin into the dH2O which the dialysis membrane was spinning in. Water samples were taken to test if any drug was lost at the 4 hour mark and again at the 24 hour mark. In result of these experiments, it was observed that a 2 kDa dialysis membrane was an effective tool to retain approximately 3 kDa Nisin molecules while removing the smaller molecules (e.g., NaCl, which had an approximate size of about 0.058 kDa). To remove additional contaminants, additional filtration is performed to purify Nisin Z.

Taken together, these results demonstrated that various Nisins e.g., in shrimp feed compositions described herein can be used to reduce pathogenesis and promote survival of shrimp.

### Example 5. EDTA can be Used to Reduce Pathogenesis and Promote Survival of Shrimp

This Example describes the toxicity and efficacy of EDTA in reducing pathogenesis and promoting survival of shrimp. Brine shrimp (Artemia salina) were utilized as a model organism to simulate potential impacts on Pacific whiteleg shrimp (Penaeus vannamei).

### Materials and Methods

### Materials

Artemia salina (a species of brine shrimp) were used. Additional materials included: ethylenediaminetetraacetic acid (EDTA; 99% pure) powder (USB Corporation Instant Ocean (Instant Ocean Spectrum Brands. Blacksburg, VA); and Artemia salina eggs were used.

### Methods

### Brine Shrimp Cultivation and Exposure

Artemia salina eggs were hatched in artificial seawater (Instant Ocean) under controlled conditions. Newly hatched brine shrimp were then exposed to varying concentrations of EDTA in experimental tanks. A separate set of tanks was used for each concentration of EDTA (20 mM, 400 uM, 200 uM, and 100 uM).

### Experimental Design

### Control Setup

Control tanks treated with saline water were prepared to observe the natural behavior and survival rate of Artemia salina without EDTA exposure.

### Treatment Groups

Brine shrimp were divided into different tanks for exposure to varying concentrations (20 mM, 400 uM, 200 uM, and 100 uM) of EDTA.

### Data Collection

Mortality and behavior of brine shrimp were recorded at 24 and 48 hours post-exposure.

### Results

EDTA at concentration 20 mM was found to have 100% mortality at both 24 and 48 hours post-exposure, but at concentrations less than or equal to 400 M, zero mortality was observed at 24 and 48 hours post-exposure.

Taken together, these results demonstrated that EDTA can be added to shrimp feed compositions comprising Nisin described herein to reduce pathogenesis and promote survival of shrimp.

EDTA is a chelating agent that binds divalent metal ions such as Ca²⁺ and Mg²⁺ which are essential for bacterial cell wall and membrane integrity. Gram-negative bacteria have an outer membrane barrier (gram-positive bacteria do not) which makes them more resistant to Nisin. Without being bound by theory, EDTA may destabilize the outer membrane in gram-negative bacteria, allowing Nisin better access to the inner membrane where it can form pores.

### Example 6, Nisin and EDTA Act Synergistically to Reduce Pathogenesis and Promote Survival of Shrimp

This Example demonstrated the combined efficacy of Nisin and EDTA against various pathogenic bacteria, including Vibrio species, Staphylococcus spp., and E. coli, focusing on the zones of inhibition (ZOI) indicative of bacterial growth reduction.

### Materials and Methods

### Materials

ImmuCell Nisin A (96% pure) suspended in citric acid buffer (pH 3.4) at 4.05 mg/mL (ImmuCell, Portland, ME); Nisin A (Nissen®-A; 10% pure powder, Siveele, Breda, Netherlands): Citric Acid Buffer (CAB) pH=3.4; 60 mm Petri Dishes; Laboratory control strain of Staphylococcus aureus (“SAI”), experimental strains of Escherichia coli (“ECY” and “ECX”) and Vibrio parahaemolyticus (“VPY”, and “VPX”); and ethylenediaminetetraacetic acid (EDTA: 99% pure) powder (USB Corporation) were used.

### Bacterial Culture Preparation

On the day before the experiment, stocks of Vibrio parahaemolyticus, Staphylococcus aureus, and Escherichia coli were prepared. An inoculation loop was sterilized, cooled, and used to extract cells, which were then cultured in nutrient broth at 37° C. for 16 to 20 hours. The bacterial density was adjusted to approximately 10⁷ to 10⁸ CFU/mL. Subsequently, 100 μL of each culture, normalized to about 10⁶ to 10⁷ CFU/mL, was evenly spread on agar plates using sterile glass beads, creating a uniform bacterial lawn. Four identical lawns were prepared for each bacterial type to ensure replicative testing of treatments.

### Nisin and EDTA Treatments

Two forms of Nisin, 96% pure Nisin (Immucell) and 10% pure Niseen (Sivele), were prepared in citric acid buffer (CAB) at a concentration of 4.05 mg/mL. To assess synergistic effects, EDTA was added to each Nisin solution to achieve a final concentration of 20.8 mM.

### Application and Incubation

5 mL of each treatment formulation were spotted onto the prepared bacterial lawns. These plates were then incubated at 37° C. for 16 to 20 hours to allow for bacterial growth and ZOI formation.

### Results

The ZOI were observed and measured in millimeters to measure the degree of bacterial growth suppression. A comparative analysis was conducted between treatments with and without EDTA, Nisin's activity was enhanced by the inclusion of EDTA, with the 96% pure Immucell Nisin+EDTA group showing a minimum increase in ZOI diameter against gram-negative experimental strains of 16.67% and a maximum of 50%, as compared to 7.69% for the gram-positive control strain SAL. It was also observed that the impact of EDTA on 10% pure Nisin varied, with some cases showing no change and others demonstrating significant inhibition of a strain not sensitive to Nisin on its own (i.e., VPY).

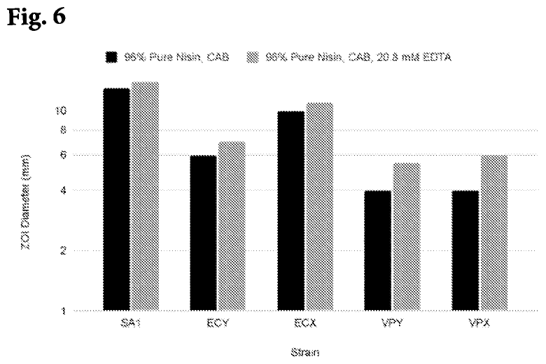

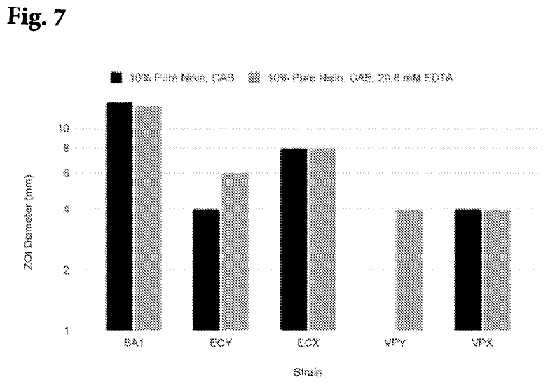

The results are provided in FIG. 6, FIG. 7, and Table 8, demonstrating that Nisin and EDTA acted synergistically to reduce pathogenesis and promote survival of shrimp.

TABLE 8

| | | | Diameter | Percent Increase |
|---|---|---|---|---|
| Stain | Strain | Treatment | (mm) | w/ EDTA |
| Gram | SA1 | 4.05 mg/mL 96% pure Nisin in CAB | 13 | 7.69% |
| positive | | 4.05 mg/mL 96% pure Nisin in CAB + | 14 | |
| | | 20.8 mM EDTA | | |
| | | 4.05 mg/mL 10% pure Nisin in CAB | 13 | 0.00% |
| | | 4.05 mg/mL 10% pure Nisin in CAB + | 13 | |
| | | 20.8 mM EDTA | | |
| Gram | ECY | 4.05 mg/mL 96% pure Nisin in CAB | 6 | 16.67% |
| negative | | 4.05 mg/mL 96% pure Nisin in CAB + | 7 | |
| | | 20.8 mM EDTA | | |
| | | 4.05 mg/mL 10% pure Nisin in CAB | 4 | 50.00% |
| | | 4.05 mg/mL 10% pure Nisin in CAB + | 6 | |
| | | 20.8 mM EDTA | | |
| | ECX | 4.05 mg/mL 96% pure Nisin in CAB | 10 | 10.00% |
| | | 4.05 mg/mL 96% pure Nisin in CAB + | 11 | |
| | | 20.8 mM EDTA | | |
| | | 4.05 mg/mL 10% pure Nisin in CAB | 8 | 0.00% |
| | | 4.05 mg/mL 10% pure Nisin in CAB + | 8 | |
| | | 20.8 mM EDTA | | |
| | VPY | 4.05 mg/mL 96% pure Nisin in CAB | 4 | 25.00% |
| | | 4.05 mg/mL 96% pure Nisin in CAB + | 5 | |
| | | 20.8 mM EDTA | | |
| | | 4.05 mg/mL 10% pure Nisin in CAB | 0 | 100.00% |
| | | 4.05 mg/mL 10% pure Nisin in CAB + | 4 | |
| | | 20.8 mM EDTA | | |
| | VPX | 4.05 mg/mL 96% pure Nisin in CAB | 4 | 50.00% |
| | | 4.05 mg/mL 96% pure Nisin in CAB + | 6 | |
| | | 20.8 mM EDTA | | |
| | | 4.05 mg/mL 10% pure Nisin in CAB | 4 | 0.00% |
| | | 4.05 mg/mL 10% pure Nisin in CAB + | 4 | |
| | | 20.8 mM EDTA | | |

Abbreviations: SA1 = Staphylococcus aureus (a control strain);
ECY = E. Coli (experimental strain with low sensitivity to Nisin);
ECX = E. Coli (experimental strain with high sensitivity to Nisin);
VPY = Vibrio parahaemolyticus (experimental strain with low sensitivity to Nisin); and
VPX = Vibrio parahaemolyticus (experimental strain with high sensitivity to Nisin).

EDTA is a chelating agent that binds divalent metal ions such as Ca²⁺ and Mg²⁺ which are essential for bacterial cell wall and membrane integrity. Gram-negative bacteria have an outer membrane barrier (gram-positive bacteria do not), which makes them more resistant to Nisin. Without being bound by theory, EDTA may destabilize the outer membrane in gram-negative bacteria, allowing Nisin better access to the inner membrane where it can form pores. Counter to the expected results, these experiments described herein showed that EDTA provides variable degrees of synergistic activity with Nisin in a purity-dependent fashion. Furthermore, the results demonstrated that EDTA is capable of not only improving the efficacy of Nisin, but also expanding the antimicrobial coverage of low purity Nisin by making previously-insensitive strains of Vibrio parahaemolyticus, sensitive (see e.g., FIG. 7, VPY group). These results have substantial implications in shrimp aquaculture where low purity Nisin is necessitated over high purity, due to economic constraints. In particular, these results demonstrate that monetarily cheaper, low purity Nisin with EDTA to be used instead of more expensive, high purity Nisin, without the loss of antimicrobial efficacy.

### Example 7. Brine Shrimp Efficacy Challenge Trial with and without Nisin

Brine shrimp (Artemia salina) are used and are prepared according to methods detailed in Examples 1-6. Additional materials, methods and experimental design may be similar to that described in Examples 1-6. A virulent strain of Vibrio parahaemolyticus is cultured to a concentration of about 1.2⁶ CFU/mL, which is used for the challenge. Shrimp groups are divided: one group receives no treatment (as control) and the other group set receives brine infused with Nisin Z (concentration as determined optimal in prior experiments).

The control group of brine shrimp, exposed to Vibrio parahaemolyticus without Nisin Z treatment, exhibit high mortality rates due to the virulence of the bacterial strain. Conversely, the group treated with Nisin Z show significantly lower mortality rates, demonstrating the efficacy of Nisin Z in reducing Vibrio-related fatalities in brine shrimp through antimicrobial activity.

Taken together, these results demonstrate that Nisin Z confers protective effects against Vibrio infections in marine aquaculture environments, further demonstrating its utility as a preventative treatment in commercial aquaculture operations, such as in compositions comprising shrimp feed and a bacteriocin described herein.

### Example 8. Vibrio Killing Concentration Assay

Utilizing materials, methods and experimental designs as described in Examples 1-6, various concentrations of Vibrio parahaemolyticus (1.2⁶ CFU/mL, 1.2⁷ CFU/mL, and 1.2⁸ CFU/mL) are introduced to separate tanks of brine shrimp to determine the killing control concentration.

A dose-dependent increase in mortality among the brine shrimp is observed, with the highest concentration exhibiting near or total lethality. This assay confirms the minimum bacterial concentration necessary to achieve 100% mortality, serving as a control measure for subsequent antimicrobial testing.

These results establish the lethal dose of Vibrio necessary for effective in vivo testing of antibacterial agents such as Nisin in brine shrimp, facilitating accurate assessments of potential treatments.

### Example 9. Pre- and Post-Dialysis Toxicity Assay and Nisin/EDTA Toxicity Assay

Utilizing materials, methods and experimental designs as described in Examples 1-6, Nisin Z is subjected to a 48-hour dialysis treatment to reduce impurities. The toxicity assay involves exposing brine shrimp to treated and untreated samples of Nisin Z, both alone and in combination with EDTA at a concentration of 400 PM.

The post-dialyzed Nisin Z shows reduced toxicity due to the removal of impurities. When combined with EDTA, the formulation exhibits minimal-to-no toxicity at the tested concentration, aligning with the individual non-toxic profiles of each component (see Example 4 and Example 5).

These results demonstrate that dialysis effectively reduces the toxicity of Nisin Z, facilitated by the removal of impurities and making it safer for broader application. Additionally, the synergistic non-toxic combination of Nisin Z and EDTA offers improved protective effects against Vibrio infections in marine aquaculture environments.

### INCORPORATION BY REFERENCE

All publications and patents cited throughout the text of this specification (including all patents, patent applications, scientific publications, manufacturer's specifications, instructions, etc.), whether supra or infra, are hereby incorporated by reference in their entirety for all purposes. To the extent the material incorporated by reference contradicts or is inconsistent with this specification, the specification will supersede any such material.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A composition comprising:
a bacteriocin, wherein the bacteriocin comprises Nisin; and
shrimp feed,
wherein the composition prevents or treats a disease or one or more symptoms thereof caused by pathogenic Vibrio in a shrimp in need thereof.

2. The composition of claim 1, wherein the shrimp feed comprises: one or more of proteins, amino acids, carbohydrates and/or starches, vitamins and/or minerals, fatty acids and/or phospholipids, non-nutrient additives, and combinations thereof.

3. The composition of claim 2, wherein:
a. the proteins comprise: fish meal, soybean meal, shrimp meal, sardine meal, poultry meal, squid meal, corn gluten meal, krill meal, insect meal, and protein from a single-cell source;
b. the amino acids comprise: methionine, threonine, lysine, arginine, valine, histidine, phenylalanine, and tryptophan;
c. the carbohydrates and/or starches comprise: wheat flour, wheat gluten meal, corn, and rice;
d. the vitamins and/or minerals comprise: pre-mix, Ca(H₂PO₄)₂ with vitamin C, choline chlorine with chloride, salt, potassium chloride, calcium carbonate, magnesium sulfate, and monosodium phosphate:
e. the fatty acids and/or phospholipids comprise: fish oil, tuna oil, squid oil, krill oil, algae oil, soy lecithin oil, and vegetable oil; and/or
f. the non-nutrient additives comprise: a binder, soy lecithin, and a mold inhibitor.

4. The composition of claim 3, wherein:
a. the binder comprises carboxymethylcellulose; and/or
b. the mold inhibitor comprises calcium propionate.

5. The composition of any one of claims 2-4, wherein the shrimp feed comprises: proteins, amino acids, carbohydrates and starches, vitamins and minerals, fatty acids and phospholipids, and non-nutrient additives.

6. The composition of claim 5, wherein the:
a. proteins and amino acids comprise: from 20% to 65% wt/v of the composition;
b. carbohydrates and starches comprise: from 15% to 40% wt/v of the composition;
c. vitamins and minerals comprise: from 0% to 10% wt/v of the composition;
d. fatty acids and phospholipids comprise: from 0% to 10% wt/v of the composition; and
e. non-nutrient additives comprise: from 0% to 10% wt/v of the composition.

7. The composition of any one of the preceding claims, wherein the shrimp feed comprises: anchovy meal, soybean meal, fish oil, tuna oil, wheat flour, krill meal, wheat gluten meal, choline chloride with choline, cholesterol, soy lecithin, Ca(H₂PO₄)₂, salt, Rovimix-stay-C 35, shrimp pre-mix, methionine, threonine, lysine, carboxymethylcellulose, and calcium propionate.

8. The composition of claim 7, wherein the:
a. anchovy meal comprises about 26.5% wt/v of the shrimp feed:
b. soybean meal comprises about 30.4% wt/v of the shrimp feed:
c. fish oil and tuna oil comprise about 1.8% wt/v of the shrimp feed;
d. wheat flour comprises about 27.27% wt/v of the shrimp feed;
e. krill meal comprises about 2% wt/v of the shrimp feed;
f. wheat gluten meal comprises about 3.5% wt/v of the shrimp feed;
g. choline chloride with choline comprises about 1% wt/v of the shrimp feed;
h. cholesterol comprises about 0.03% wt/v of the shrimp feed;
i. soy lecithin comprises about 1.5% wt/v of the shrimp feed;
j. Ca(H₂PO₄)₂ comprises about 2% wt/v of the shrimp feed;
k. salt comprises about 0.6% wt/v of the shrimp feed;
l. Rovimix-stay-C 35 comprises about 0.1% wt/v of the shrimp feed;
m. shrimp pre-mix comprises about 0.4% wt/v of the shrimp feed;
n. methionine comprises about 0.2% wt/v of the shrimp feed;
o. threonine comprises about 0.3% wt/v of the shrimp feed;
p. lysine comprises about 0.3% wt/v of the shrimp feed;
q. carboxymethylcellulose comprises about 2% wt/v of the shrimp feed; and
r. calcium propionate comprises about 0.1% wt/v of the shrimp feed.

9. The composition of any one of the preceding claims, wherein the bacteriocin comprises from about 0.0001% to 20% wt/v of the composition.

10. The composition of any one of the preceding claims, wherein the bacteriocin comprises from about 1×10⁻³ mg/g to 200 mg/g of the composition.

11. The composition of any one of the preceding claims, wherein the Nisin comprises from about 0.0001% to 10% wt/v of the composition.

12. The composition of any one of the preceding claims, wherein the:
a. shrimp feed comprises about 99.99% wt/v of the composition; and
b. the bacteriocin comprises about 0.01% w/v of the composition.

13. The composition of any one of the preceding claims, wherein the Nisin comprises Nisin A, Nisin E, Nisin F, Nisin G, Nisin H, Nisin J, Nisin O, Nisin P, Nisin Q, Nisin U, Nisin U2, Nisin P, Nisin Z, combinations thereof, and engineered variants thereof.

14. The composition of claim 113, wherein the Nisin comprises Nisin Z.

15. The composition of any one of claims 1-11 and 13-14, wherein the composition further comprises ethylenediaminetetraacetic acid (EDTA).

16. The composition of claim 15, wherein the EDTA comprises from about 0.00010% to 2% wt/v of the composition.

17. The composition of claim 15 or 16, wherein the EDTA comprises from about 0.001 mg/g to 20 mg/g of the composition.

18. The composition of any one of claims 15-17, wherein the:
a. shrimp feed comprises about 99.98% wt/v of the composition;
b. the bacteriocin comprises about 0.010% w/v of the composition; and
c. the EDTA comprises about 0.01% wt/v of the composition.

19. A composition comprising:
a bacteriocin, wherein the bacteriocin comprises Nisin Z, and wherein the bacteriocin comprises about 0.01% w/v of the composition; and
shrimp feed, wherein the shrimp feed comprises about 99.99% wt/v of the composition, and wherein:
a. anchovy meal comprises about 26.5% wt/v of the shrimp feed;
b. soybean meal comprises about 30.4% wt/v of the shrimp feed;
c. fish oil and tuna oil comprise about 1.8% wt/v of the shrimp feed;
d. wheat flour comprises about 27.27% N wt/v of the shrimp feed;
e. krill meal comprises about 2% wt/v of the shrimp feed;
f. wheat gluten meal comprises about 3.5% wt/v of the shrimp feed;
g. choline chloride with choline comprises about 1% wt/v of the shrimp feed;
h. cholesterol comprises about 0.03% wt/v of the shrimp feed;
i. soy lecithin comprises about 1.5% wt/v of the shrimp feed;
j. Ca(H₂PO₄)₂ comprises about 2% wt/v of the shrimp feed;
k. salt comprises about 0.6% wt/v of the shrimp feed;
l. Rovimix-stay-C 35 comprises about 0.1% wt/v of the shrimp feed;
m. shrimp pre-mix comprises about 0.4% wt/v of the shrimp feed;
n. methionine comprises about 0.2% wt/v of the shrimp feed;
o. threonine comprises about 0.3% v wt/v of the shrimp feed;
p. lysine comprises about 0.3% wt/v of the shrimp feed;
q. carboxymethylcellulose comprises about 2% wt/v of the shrimp feed; and
r. calcium propionate comprises about 0.1% wt/v of the shrimp feed,
wherein the composition prevents or treats a disease or one or more symptoms thereof caused by pathogenic Vibrio in a shrimp in need thereof.

20. A composition comprising:
a bacteriocin, wherein the bacteriocin comprises Nisin Z, and wherein the bacteriocin comprises about 0.01% w/v of the composition;
EDTA, wherein the EDTA comprises about 0.01% wt/v of the composition; and
shrimp feed, wherein the shrimp feed comprises about 99.98% wt/v of the composition, and wherein:
a. anchovy meal comprises about 26.5% wt/v of the shrimp feed;
b. soybean meal comprises about 30.4% wt/v of the shrimp feed;
c. fish oil and tuna oil comprise about 1.8% wt/v of the shrimp feed;
d. wheat flour comprises about 27.27% wt/v of the shrimp feed;
e. krill meal comprises about 2% wt/v of the shrimp feed;
f. wheat gluten meal comprises about 3.5% wt/v of the shrimp feed;
g. choline chloride with choline comprises about 1% wt/v of the shrimp feed;
h. cholesterol comprises about 0.03% wt/v of the shrimp feed;
i. soy lecithin comprises about 1.5% wt/v of the shrimp feed;
j. Ca(H₂PO₄)₂ comprises about 2% wt/v of the shrimp feed;
k. salt comprises about 0.6% wt/v of the shrimp feed;
l. Rovimix-stay-C 35 comprises about 0.1% wt/v of the shrimp feed;
m. shrimp pre-mix comprises about 0.4% wt/v of the shrimp feed;
n. methionine comprises about 0.2% wt/v of the shrimp feed;
o. threonine comprises about 0.3% wt/v of the shrimp feed;
p. lysine comprises about 0.3% wt/v of the shrimp feed;
q. carboxymethylcellulose comprises about 2% wt/v of the shrimp feed; and
r. calcium propionate comprises about 0.1% wt/v of the shrimp feed,
wherein the composition prevents or treats a disease or one or more symptoms thereof caused by pathogenic Vibrio in a shrimp in need thereof.

21. The composition of any one of the preceding claims, wherein the bacteriocin further comprises: a Class I, Class II, and/or Class III bacteriocin.

22. The composition of claim 21, wherein the Class I, Class II, and/or Class III bacteriocin comprises a colicin, colicin-like bacteriocin, klebicin, microcin, or phage-tail-like bacteriocin.

23. The composition of any one of the preceding claims, wherein the bacteriocin and shrimp feed are combined.

24. The composition of claim 23, wherein combined comprises: top-coating, extrusion, or mixing-in.

25. The composition of any one of the preceding claims, wherein the composition is administered to the shrimp in need thereof.

26. The composition of claim 25, wherein the shrimp in need thereof is or has not been administered an antibiotic.

27. The composition of claim 25 or 26, wherein upon administration, a concentration of pathogenic Vibrio is reduced in the shrimp.

28. The composition of any one of claims 25-27, wherein upon administration, the one or more symptoms thereof is reduced in the shrimp.

29. The composition of claim 28, wherein the one or more symptoms comprise lethargy, anorexia, slow growth, an empty digestive tract, pale-to-white hepatopancreases, reddish body discoloration, yellowing gill tissue, appearance of white patches in abdominal muscle, melanisation, granulomatous encapsulation, luminescence, a soft shell, melanised spots or streaks visible within the hepatopancreas, and atrophy of the hepatopancreas.

30. The composition of any one of claims 25-29, wherein upon administration, a microbiome of the shrimp is modified relative to a suitable control.

31. The composition of any one of the preceding claims, wherein the pathogenic Vibrio comprise Vibrio parahaemolyticus, Vibrio alginolyticus, Vibrio vulnificus, Vibrio cholera, Vibrio harveyi, Vibrio fischeri, Vibrio anguillarum, Vibrio spledidus, Vibrio nerves, Vibrio tubiashii, Vibrio campbellii, Vibrio aestuarianus, Vibrio pelagius, Vibrio mediterranei, Vibrio diazotrophicus, Vibrio damselae, Vibrio mimicus, and Vibrio cholera.

32. The composition of any one of the preceding claims, wherein the disease comprises: acute hepatopancreatic necrosis disease (AHPND), vibriosis, loose shell syndrome (LSS), tail necrosis, shell disease, red disease, white gut disease (WGD), white tail disease, white feces disease (WFD), luminous bacterial disease, and necrotizing hepatopancreatitis.

33. The composition of any one of the preceding claims, wherein the composition does not comprises an antibiotic.

34. The composition of any one of the preceding claims, wherein the shrimp in need thereof is a farmed shrimp.

35. A method of preventing or treating a disease or one or more symptoms thereof caused by pathogenic Vibrio in a shrimp in need thereof, the method comprising administering the composition of any one of claims 1-34.

36. A method of reducing a concentration of pathogenic Vibrio in a shrimp in need thereof, the method comprising administering the composition of any one of claims 1-34.

37. A method of preventing or treating a disease or one or more symptoms thereof caused by pathogenic Vibrio in a shrimp in need thereof, the method comprising
administering a composition comprising:
a bacteriocin, wherein the bacteriocin comprises Nisin; and
shrimp feed.

38. A method of preventing or treating a disease or one or more symptoms thereof caused by pathogenic Vibrio in a shrimp in need thereof, the method comprising
administering a composition comprising:
a bacteriocin, wherein the bacteriocin comprises Nisin;
shrimp feed; and
EDTA.

39. A method of reducing a concentration of pathogenic Vibrio in a shrimp in need thereof, the method comprising administering a composition comprising:
a bacteriocin, wherein the bacteriocin comprises Nisin; and
shrimp feed.

40. A method of reducing a concentration of pathogenic Vibrio in a shrimp in need thereof, the method comprising administering a composition comprising:
a bacteriocin, wherein the bacteriocin comprises Nisin;
shrimp feed; and
EDTA.

41. The method of claim 39 or 40, wherein the shrimp feed comprises: one or more of proteins, amino acids, carbohydrates and/or starches, vitamins and/or minerals, fatty acids and/or phospholipids, non-nutrient additives, and combinations thereof.

42. The method of claim 41, wherein:
a. the proteins comprise: fish meal, soybean meal, shrimp meal, sardine meal, poultry meal, squid meal, corn gluten meal, krill meal, insect meal, and protein from a single-cell source;
b. the amino acids comprise: methionine, threonine, lysine, arginine, valine, histidine, phenylalanine, and tryptophan;
c. the carbohydrates and/or starches comprise: wheat flour, wheat gluten meal, corn, and rice;
d. the vitamins and/or minerals comprise: pre-mix, Ca(H₂PO₄)₂ with vitamin C, choline chlorine with chloride, salt, potassium chloride, calcium carbonate, magnesium sulfate, and monosodium phosphate;
e. the fatty acids and/or phospholipids comprise: fish oil, tuna oil, squid oil, krill oil, algae oil, soy lecithin oil, and vegetable oil; and/or
f. the non-nutrient additives comprise: a binder, soy lecithin, and a mold inhibitor.

43. The method of claim 42, wherein:
a. the binder comprises carboxymethylcellulose; and/or
b. the mold inhibitor comprises calcium propionate.

44. The method of any one of claims 41-43, wherein the shrimp feed comprises: proteins, amino acids, carbohydrates and starches, vitamins and minerals, fatty acids and phospholipids, and non-nutrient additives.

45. The method of claim 44, wherein the:
a. proteins and amino acids comprise: from 20% to 65% wt/v of the composition;
b. carbohydrates and starches comprise: from 15% to 40% wt/v of the composition;
c. vitamins and minerals comprise: from 0% to 10% wt/v of the composition;
d. fatty acids and phospholipids comprise: from 0% to 10% wt/v of the composition; and
e. non-nutrient additives comprise: from 0% to 10% wt/v of the composition.

46. The method of any one of claims 39-46, wherein the shrimp feed comprises: anchovy meal, soybean meal, fish oil, tuna oil, wheat flour, krill meal, wheat gluten meal, choline chloride with choline, cholesterol, soy lecithin, Ca(H₂PO₄)₂, salt, Rovimix-stay-C 35, shrimp pre-mix, methionine, threonine, lysine, carboxymethylcellulose, and calcium propionate.

47. The method of claim 46, wherein the:
a. anchovy meal comprises about 26.5% wt/v of the shrimp feed;
b. soybean meal comprises about 30.4% wt/v of the shrimp feed;
c. fish oil and tuna oil comprise about 1.8% wt/v of the shrimp feed;
d. wheat flour comprises about 27.27% wt/v of the shrimp feed;
e. krill meal comprises about 2% wt/v of the shrimp feed;
f. wheat gluten meal comprises about 3.5% v wt/v of the shrimp feed;
g. choline chloride with choline comprises about 1% wt/v of the shrimp feed;
h. cholesterol comprises about 0.03% wt/v of the shrimp feed;
i. soy lecithin comprises about 1.5% wt/v of the shrimp feed;
j. Ca(H₂PO₄)₂ comprises about 2% wt/v of the shrimp feed;
k. salt comprises about 0.6% wt/v of the shrimp feed;
l. Rovimix-stay-C 35 comprises about 0.1% wt/v of the shrimp feed;
m. shrimp pre-mix comprises about 0.4% wt/v of the shrimp feed;
n. methionine comprises about 0.2% wt/v of the shrimp feed;
o. threonine comprises about 0.3% wt/v of the shrimp feed;
p. lysine comprises about 0.3% wt/v of the shrimp feed;
q. carboxymethylcellulose comprises about 2% wt/v of the shrimp feed; and
r. calcium propionate comprises about 0.1% wt/v of the shrimp feed.

48. The method of any one of claims 39-47, wherein the bacteriocin comprises from about 0.0001% to 20% wt/v of the composition.

49. The method of any one of claims 39-48, wherein the bacteriocin comprises from about 1×10³ mg/g to 200 mg/g of the composition.

50. The method of any one of claims 39-49, wherein the Nisin comprises from about 0.0001% to 10% wt/v of the composition.

51. The method of any one of claims 39-50, wherein the:
a. shrimp feed comprises about 99.99% wt/v of the composition; and
b. the bacteriocin comprises about 0.01% w/v of the composition.

52. The method of any one of claims 39-51, wherein the Nisin comprises Nisin A, Nisin E, Nisin F, Nisin G, Nisin H, Nisin J, Nisin O, Nisin P, Nisin Q, Nisin U, Nisin U2, Nisin P, Nisin Z, combinations thereof, and engineered variants thereof.

53. The method of claim 51, wherein the Nisin comprises Nisin Z.

54. The method of any one of claims 39-50 and 52-53, wherein the composition further comprises ethylenediaminetetraacetic acid (EDTA).

55. The method of claim 54, wherein the EDTA comprises from about 0.0001% to 2% wt/v of the composition.

56. The method of claim 54 or 55, wherein the EDTA comprises from about 0.001 mg/g to 20 mg/g of the composition.

57. The method of any one of claims 54-56, wherein the:
a. shrimp feed comprises about 99.98% wt/v of the composition;
b. the bacteriocin comprises about 0.010% w/v of the composition; and
c. the EDTA comprises about 0.01% wt/v of the composition.

58. The method of claim 39 or 40, wherein the composition comprises:
a bacteriocin, wherein the bacteriocin comprises Nisin Z, and wherein the bacteriocin comprises about 0.01% w/v of the composition; and
shrimp feed, wherein the shrimp feed comprises about 99.99% wt/v of the composition, and wherein:
a. anchovy meal comprises about 26.5% wt/v of the shrimp feed;
b. soybean meal comprises about 30.4% wt/v of the shrimp feed;
c. fish oil and tuna oil comprise about 1.8% wt/v of the shrimp feed;
d. wheat flour comprises about 27.27% wt/v of the shrimp feed;
e. krill meal comprises about 2% wt/v of the shrimp feed;
f. wheat gluten meal comprises about 3.5% wt/v of the shrimp feed;
g. choline chloride with choline comprises about 1% wt/v of the shrimp feed;
h. cholesterol comprises about 0.03% wt/v of the shrimp feed;
i. soy lecithin comprises about 1.5% wt/v of the shrimp feed;
j. Ca(H₂PO₄)₂ comprises about 2% wt/v of the shrimp feed;
k. salt comprises about 0.6% wt/v of the shrimp feed;
l. Rovimix-stay-C 35 comprises about 0.1% wt/v of the shrimp feed;
m. shrimp pre-mix comprises about 0.4% wt/v of the shrimp feed;
n. methionine comprises about 0.2% wt/v of the shrimp feed;
o. threonine comprises about 0.3% wt/v of the shrimp feed;
p. lysine comprises about 0.3% wt/v of the shrimp feed;
q. carboxymethylcellulose comprises about 2% wt/v of the shrimp feed; and
r. calcium propionate comprises about 0.10% wt/v of the shrimp feed.

59. The method of claim 39 or 40, wherein the composition comprises:
a bacteriocin, wherein the bacteriocin comprises Nisin Z, and wherein the bacteriocin comprises about 0.01% w/v of the composition;
EDTA, wherein the EDTA comprises about 0.01% wt/v of the composition; and
shrimp feed, wherein the shrimp feed comprises about 99.98% wt/v of the composition, and wherein:
a. anchovy meal comprises about 26.5% wt/v of the shrimp feed;
b. soybean meal comprises about 30.4% wt/v of the shrimp feed;
c. fish oil and tuna oil comprise about 1.8% wt/v of the shrimp feed;
d. wheat flour comprises about 27.27% wt/v of the shrimp feed;
e. krill meal comprises about 2% wt/v of the shrimp feed;
f. wheat gluten meal comprises about 3.5% wt/v of the shrimp feed;
g. choline chloride with choline comprises about 1% wt/v of the shrimp feed;
h. cholesterol comprises about 0.03% wt/v of the shrimp feed;
i. soy lecithin comprises about 1.5% wt/v of the shrimp feed;
j. Ca(H₂PO₄)₂ comprises about 2% wt/v of the shrimp feed;
k. salt comprises about 0.6% wt/v of the shrimp feed;
l. Rovimix-stay-C 35 comprises about 0.1% wt/v of the shrimp feed;
m. shrimp pre-mix comprises about 0.4% wt/v of the shrimp feed;
n. methionine comprises about 0.2% wt/v of the shrimp feed;
o. threonine comprises about 0.3% wt/v of the shrimp feed;
p. lysine comprises about 0.3% wt/v of the shrimp feed;
q. carboxymethylcellulose comprises about 2% wt/v of the shrimp feed; and
r. calcium propionate comprises about 0.1% wt/v of the shrimp feed.

60. The method of any one of claims 39-59, wherein the bacteriocin further comprises: a Class I, Class II, and/or Class III bacteriocin.

61. The method of claim 60, wherein the Class I, Class II, and/or Class III bacteriocin comprises a colicin, colicin-like bacteriocin, klebicin, microcin, or phage-tail-like bacteriocin.

62. The method of any one of claims 39-61, wherein the bacteriocin and shrimp feed are combined.

63. The method of claim 62, wherein combined comprises: top-coating, extrusion, or mixing-in.

64. The method of claim 63, wherein the shrimp in need thereof is or has not been administered an antibiotic.

65. The method of any one of claims 39-64, wherein upon administration, a concentration of pathogenic Vibrio is reduced in the shrimp.

66. The method of any one of claims 39-65, wherein upon administration, one or more diseases caused by pathogenic Vibrio or symptoms thereof in the shrimp are reduced, prevented, or treated.

67. The method of claim 66, wherein the one or more symptoms comprise lethargy, anorexia, slow growth, an empty digestive tract, pale-to-white hepatopancreases, reddish body discoloration, yellowing gill tissue, appearance of white patches in abdominal muscle, melanisation, granulomatous encapsulation, luminescence, a soft shell, melanised spots or streaks visible within the hepatopancreas, and atrophy of the hepatopancreas.

68. The method of any one of claims 39-67, wherein upon administration, a microbiome of the shrimp is modified relative to a suitable control.

69. The method of any one of claims 39-68, wherein the pathogenic Vibrio comprise Vibrio parahaemolyticus, Vibrio alginolyticus, Vibrio vulnificus, Vibrio cholera, Vibrio harveyi, Vibrio fischeri, Vibrio anguillarum, Vibrio spledidus, Vibrio nerves, Vibrio tubiashii, Vibrio campbellii, Vibrio aestuarianus, Vibrio pelagius, Vibrio mediterranei, Vibrio diazotrophicus, Vibrio damselae, Vibrio mimicus, and Vibrio cholera.

70. The method of any one of claims 39-69, wherein the disease comprises: acute hepatopancreatic necrosis disease (AHPND), vibriosis, loose shell syndrome (LSS), tail necrosis, shell disease, red disease, white gut disease (WGD), white tail disease, white feces disease (WFD), luminous bacterial disease, and necrotizing hepatopancreatitis.

71. The method of any one of claims 39-70, wherein the composition does not comprises an antibiotic.

72. The method of any one of claims 39-71, wherein the shrimp in need thereof is a farmed shrimp.

73. A method of reducing a concentration of pathogenic Vibrio in a shrimp in need thereof, the method comprising administering a composition comprising:
a bacteriocin, wherein the bacteriocin comprises Nisin Z, and wherein the bacteriocin comprises about 0.01% w/v of the composition; and
shrimp feed, wherein the shrimp feed comprises about 99.99% wt/v of the composition, and wherein:
a. anchovy meal comprises about 26.5% N wt/v of the shrimp feed;
b. soybean meal comprises about 30.4% wt/v of the shrimp feed;
c. fish oil and tuna oil comprise about 1.8% wt/v of the shrimp feed;
d. wheat flour comprises about 27.27% wt/v of the shrimp feed;
e. krill meal comprises about 2% wt/v of the shrimp feed;
f. wheat gluten meal comprises about 3.5% wt/v of the shrimp feed;
g. choline chloride with choline comprises about 1% wt/v of the shrimp feed;
h. cholesterol comprises about 0.03% wt/v of the shrimp feed;
i. soy lecithin comprises about 1.5% wt/v of the shrimp feed;
j. Ca(H₂PO₄)₂ comprises about 2% wt/v of the shrimp feed;
k. salt comprises about 0.6% wt/v of the shrimp feed;
l. Rovimix-stay-C 35 comprises about 0.1% wt/v of the shrimp feed;
m. shrimp pre-mix comprises about 0.4% wt/v of the shrimp feed;
n. methionine comprises about 0.2% wt/v of the shrimp feed;
o. threonine comprises about 0.3% wt/v of the shrimp feed;
p. lysine comprises about 0.3% wt/v of the shrimp feed;
q. carboxymethylcellulose comprises about 2% wt/v of the shrimp feed; and
r. calcium propionate comprises about 0.1% wt/v of the shrimp feed.

74. A method of preventing or treating a disease or one or more symptoms thereof caused by pathogenic Vibrio in a shrimp in need thereof, the method comprising
administering a composition comprising:
a bacteriocin, wherein the bacteriocin comprises Nisin Z, and wherein the bacteriocin comprises about 0.01% w/v of the composition; and
shrimp feed, wherein the shrimp feed comprises about 99.99% wt/v of the composition, and wherein:
a. anchovy meal comprises about 26.5% wt/v of the shrimp feed;
b. soybean meal comprises about 30.4% wt/v of the shrimp feed;
c. fish oil and tuna oil comprise about 1.8% wt/v of the shrimp feed;
d. wheat flour comprises about 27.27% wt/v of the shrimp feed;
e. krill meal comprises about 2% wt/v of the shrimp feed;
f. wheat gluten meal comprises about 3.5% wt/v of the shrimp feed;
g. choline chloride with choline comprises about 1% wt/v of the shrimp feed;
h. cholesterol comprises about 0.03% wt/v of the shrimp feed;
i. soy lecithin comprises about 1.5% wt/v of the shrimp feed;
j. Ca(H₂PO₄)₂ comprises about 2% wt/v of the shrimp feed;
k. salt comprises about 0.6% wt/v of the shrimp feed;
l. Rovimix-stay-C 35 comprises about 0.1% wt/v of the shrimp feed;
m. shrimp pre-mix comprises about 0.4% wt/v of the shrimp feed;
n. methionine comprises about 0.2% wt/v of the shrimp feed;
o. threonine comprises about 0.3% wt/v of the shrimp feed;
p. lysine comprises about 0.3% wt/v of the shrimp feed;
q. carboxymethylcellulose comprises about 2% wt/v of the shrimp feed; and
r. calcium propionate comprises about 0.1% wt/v of the shrimp feed.

75. A method of reducing a concentration of pathogenic Vibrio in a shrimp in need thereof, the method comprising administering a composition comprising:
a bacteriocin, wherein the bacteriocin comprises Nisin Z, and wherein the bacteriocin comprises about 0.01% w/v of the composition;
EDTA, wherein the EDTA comprises about 0.01% wt/v of the composition; and
shrimp feed, wherein the shrimp feed comprises about 99.98% wt/v of the composition, and wherein:
a. anchovy meal comprises about 26.5% wt/v of the shrimp feed;
b. soybean meal comprises about 30.4% wt/v of the shrimp feed;
c. fish oil and tuna oil comprise about 1.8% wt/v of the shrimp feed;
d. wheat flour comprises about 27.27% wt/v of the shrimp feed;
e. krill meal comprises about 2% wt/v of the shrimp feed;
f. wheat gluten meal comprises about 3.5% wt/v of the shrimp feed;
g. choline chloride with choline comprises about 1% wt/v of the shrimp feed;
h. cholesterol comprises about 0.03% wt/v of the shrimp feed;
i. soy lecithin comprises about 1.5% wt/v of the shrimp feed;
j. Ca(H₂PO₄)₂ comprises about 2% wt/v of the shrimp feed;
k. salt comprises about 0.6% wt/v of the shrimp feed;
l. Rovimix-stay-C 35 comprises about 0.1% wt/v of the shrimp feed;
m. shrimp pre-mix comprises about 0.4% wt/v of the shrimp feed;
n. methionine comprises about 0.2% wt/v of the shrimp feed;
o. threonine comprises about 0.3% wt/v of the shrimp feed;
p. lysine comprises about 0.3% wt/v of the shrimp feed;
q. carboxymethylcellulose comprises about 2% wt/v of the shrimp feed; and
r. calcium propionate comprises about 0.1% wt/v of the shrimp feed.

76. A method of preventing or treating a disease or one or more symptoms thereof caused by pathogenic Vibrio in a shrimp in need thereof, the method comprising
administering a composition comprising:
a bacteriocin, wherein the bacteriocin comprises Nisin Z, and wherein the bacteriocin comprises about 0.01% w/v of the composition;
EDTA, wherein the EDTA comprises about 0.01% wt/v of the composition; and
shrimp feed, wherein the shrimp feed comprises about 99.98% wt/v of the composition, and wherein:
a. anchovy meal comprises about 26.5% wt/v of the shrimp feed;
b. soybean meal comprises about 30.4% wt/v of the shrimp feed;
c. fish oil and tuna oil comprise about 1.8% wt/v of the shrimp feed;
d. wheat flour comprises about 27.27% N wt/v of the shrimp feed;
e. krill meal comprises about 2% wt/v of the shrimp feed;
f. wheat gluten meal comprises about 3.5% wt/v of the shrimp feed;
g. choline chloride with choline comprises about 1% wt/v of the shrimp feed;
h. cholesterol comprises about 0.03% wt/v of the shrimp feed;
i. soy lecithin comprises about 1.5% wt/v of the shrimp feed;
j. Ca(H₂PO₄)₂ comprises about 2% wt/v of the shrimp feed;
k. salt comprises about 0.6% wt/v of the shrimp feed;
l. Rovimix-stay-C 35 comprises about 0.1% wt/v of the shrimp feed;
m. shrimp pre-mix comprises about 0.4% wt/v of the shrimp feed;
n. methionine comprises about 0.2% wt/v of the shrimp feed;
o. threonine comprises about 0.3% N wt/v of the shrimp feed;
p. lysine comprises about 0.3% wt/v of the shrimp feed;
q. carboxymethylcellulose comprises about 2% wt/v of the shrimp feed; and
r. calcium propionate comprises about 0.1% wt/v of the shrimp feed.
